SEQUENCE LISTING (53 sequences)

SEQ ID NO: 1 - DNA, 12 bp, Artificial Sequence
  Parallel stranded DNA (stabilised by Hoogsteen bonds or reverse Watson-Crick bonding)
cctattaaatcc

SEQ ID NO: 2 - DNA, 25 bp, Artificial Sequence
  Parallel stranded DNA (stabilised by Hoogsteen bonds or reverse Watson-Crick bonding)
aaaaaaaaaataattttaaatattt

SEQ ID NO: 3 - DNA, 10 bp, Artificial Sequence
  Hairpin
tggggcccca

SEQ ID NO: 4 - DNA, 34 bp, Artificial Sequence
  Cross-arm
atggtcttgcatgcaaggccatatatggcaccat

SEQ ID NO: 5 - DNA, 15 bp, Artificial Sequence
  Intermolecular Triplex
aagaagaagaagaag

SEQ ID NO: 6 - DNA, 43 bp, Artificial Sequence
  Triplex
cctccccctcctttttggagggggaggtttttggagggggagg

SEQ ID NO: 7 - DNA, 13 bp, Artificial Sequence
  i-motif bimolecular
cccctaaccctaa

SEQ ID NO: 8 - DNA, 26 bp, Artificial Sequence
  i-motif unimolecular
ccctaacccctaaccctaacccctaa

SEQ ID NO: 9 - DNA, 18 bp, Artificial Sequence
  Quadruplex dimeric parallel strand
aggggggagggagggtgg

SEQ ID NO: 10 - DNA, 15 bp, Artificial Sequence
  Quadruplex antiparallel unimolecular
ggttggtgtggttgg

SEQ ID NO: 11 - DNA, 12 bp, Artificial Sequence
  Quadruplex antiparallel tetramer
ttagggttaggg

SEQ ID NO: 12 - protein, 20 aa, Artificial Sequence
  NIQI
RGRGRGRGGGSGGSGGRGRG

SEQ ID NO: 13 - protein, 26 aa, Artificial Sequence
  FMR1 G-quadruplex binding protein RG-rich domain
RRGDGRRRGGGGRGQGGRGRGGGFKG

SEQ ID NO: 14 - DNA, 15 bp, Artificial Sequence
  DNA Aptamer that binds to thrombin
ggttggtgtggttgg

SEQ ID NO: 15 - DNA, 40 bp, Artificial Sequence
  Arahh001
acgtaccgacttcgtatgccaacagccctttatccacctc

SEQ ID NO: 16 - DNA, 33 bp, Artificial Sequence
  TEPP
gcgcggtaccgcgctaacggaggttgcgtccgt

SEQ ID NO: 17 - DNA, 25 bp, Artificial Sequence
  MUC1
gcagttgatcctttggataccctgg

SEQ ID NO: 18 - DNA, 72 bp, Artificial Sequence
  S1.3/2.2
gggagacaagaataaacgctcaagcagttgatcctttggataccctggttcgacaggaggctcacaacaggc

SEQ ID NO: 19 - DNA, 72 bp, Artificial Sequence
  5TR-1
gggagacaagaataaacgctcaagaagtgaaaatgacagaacacaacattcgacaggaggctcacaacaggc

SEQ ID NO: 20 - DNA, 41 bp, Artificial Sequence
  SGC8
atctaactgctgcgccgccgggaaaatactgtacggttaga

SEQ ID NO: 21 - DNA, 94 bp, Artificial Sequence
  SGA16
tttaaaataccagcttattcaattagtcacacttagagttctagctgctgcgccgccgggaaaatactgtacggatagatagtaagtgcaatct

SEQ ID NO: 22 - DNA, 48 bp, Artificial Sequence
  SYL3C
cactacagaggttgcgtctgtcccacgttgtcatggggggttggcctg

SEQ ID NO: 23 - DNA, 79 bp, Artificial Sequence
  SYL1
agcgtcgaataccactacagtttggctctgggggatgtggaggggggtatgggtgggagtcaatggagctcgtggtcag

SEQ ID NO: 24 - DNA, 80 bp, Artificial Sequence
  SYL2
agcgtcgaataccactacagagctcggggttttttggggttttttggggttttggtggggctaatggagctcgtggtcag

SEQ ID NO: 25 - DNA, 80 bp, Artificial Sequence
  SYL3
agcgtcgaataccactacagaggttgcgtctgtcccacgttgtcatggggggttggcctgctaatggagctcgtggtcag

SEQ ID NO: 26 - DNA, 81 bp, Artificial Sequence
  SYL4
agcgtcgaataccactacagagctccggggtttttgggggtttttctggggttttttggggctaatggagctcgtggtcag

SEQ ID NO: 27 - DNA, 47 bp, Artificial Sequence
  TDO5
aacaccgggaggatagttcggtggctgttcagggtctcctcccggtg

SEQ ID NO: 28 - DNA, 45 bp, Artificial Sequence
  A1
ggttgcatgccgtggggaggggggtgggttttatagcgtactcag

SEQ ID NO: 29 - DNA, 28 bp, Artificial Sequence
  AS1411
ttggtggtggtggttgtggtggtggtgg

SEQ ID NO: 30 - DNA, 75 bp, Artificial Sequence
  GMT4,8
tgacgagcccaagttaccttggtgatggtttttggtggtaacgggggcgggtgagtagaatctccgctgcctaca

SEQ ID NO: 31 - DNA, 76 bp, Artificial Sequence
  CSC1
accttggctgtcgtgttgtaggtggtttgctgcggtgggctcaagaagaaagcgcaaaggtcagtggtcagagcgt

SEQ ID NO: 32 - DNA, 77 bp, Artificial Sequence
  CSC13
accttggctgtcgtgttgtggggtgtcgtatctttcgtgtcttattattttctaggggaggtcagtggtcagagcgt

SEQ ID NO: 33 - DNA, 70 bp, Artificial Sequence
  KDED2a-3
tgcccgcgaaaactgctattacgtgtgagaggaaagatcacgcgggttcgtggacacggttttttttttt

SEQ ID NO: 34 - DNA, 91 bp, Artificial Sequence
  KCHA10
atccagagtgacgcagcaggggaggcgagagcgcacaataacgatggttgggacccaactgtttggacacggtggcttagttttttttttt

SEQ ID NO: 35 - DNA, 45 bp, Artificial Sequence
  R13
tctctagttattgagttttcttttatgggtgggtggggggttttt

SEQ ID NO: 36 - DNA, 57 bp, Artificial Sequence
  S6
tggatggggagatccgttgagtaagcgggcgtgtctctctgccgccttgctatgggg

SEQ ID NO: 37 - DNA, 69 bp, Artificial Sequence
  GBI-10
ggctgttgtgagcctcctcccagagggaagactttaggttcggttcacgtcccgcttattcttactccc

SEQ ID NO: 38 - DNA, 51 bp, Artificial Sequence
  A-1
taactcaataagctaggtgggtgggggacactacccggggggtggttgggt

SEQ ID NO: 39 - DNA, 24 bp, Artificial Sequence
  Minimal sequence to form G-quadruplex structure
ttagggttagggttagggttaggg

SEQ ID NO: 40 - DNA, 55 bp, Artificial Sequence
  no aptamer (control)
ctgctcacctgccagctacggacgcggaacgcgtccgtagctggcaggtgagcag

SEQ ID NO: 41 - DNA, 102 bp, Artificial Sequence
  H4_Gq
ctgctcacctgccagctacggacgcgtggtggggttcccgggagggcggctacgggttccgtaatcagatttgtgtcgcgtccgtagctggcaggtgagcag

SEQ ID NO: 42 - DNA, 86 bp, Artificial Sequence
  H4_SL
ctgctcacctgccagctacggacgcgcgcaggttaaatcccaaatggtccgagggttgcgcgcgtccgtagctggcaggtgagcag

SEQ ID NO: 43 - DNA, 80 bp, Artificial Sequence
  nucl
ctgctcacctgccagctacggacgcgtggtggtggtggttgtggtggtggtgggcgcgtccgtagctggcaggtgagcag

SEQ ID NO: 44 - DNA, 81 bp, Artificial Sequence
  hTEL
ctgcgcgctcgctcgctcactgaggcctttagggttagggttagggttagggttggcctcagtgagcgagcgagcgcgcag

SEQ ID NO: 45 - DNA, 2995 bp, Artificial Sequence
  Sequence of the EF1a -SEAP-SV40pA cassette
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacctaggaagcttgccaccatggttctggggccctgcatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagcagccaggatcctaaaagggcagaagaaggacaaactggggcctgagatacccctggctatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgatgtgatcctgggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggcctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccacggtcatcacgaaagcagggcttaccgggcactgactgagacgatcatgttcgacgacgccattgagagggcgggccagctcaccagcgaggaggacacgctgagcctcgtcactgccgaccactcccacgttttctccttcggaggctaccccctgcgagggagctccatcttcgggctggcccctggcaaggcacgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacgcacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccagggcggtcccggtccaagcgtctggattgagaattcgcccgggcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 46 - DNA, 181 bp, Artificial Sequence
  strSQ (4 aptamers)
ctgctcacctgccagctacggacgcggccacgaacgcaccgatcgcaggtttcgtggcgcgcgtaacgcaccgatcgcaggtttacgcgcagcgagcaacgcaccgatcgcaggtttgctcgccgcccaaacgcaccgatcgcaggttttgggcgcgcgtccgtagctggcaggtgagcag

SEQ ID NO: 47 - DNA, 78 bp, Artificial Sequence
  strApt
ctgctcacctgccagctacggacgcggggaacgcaccgatcgcaggtttccccgcgtccgtagctggcaggtgagcag

SEQ ID NO: 48 - DNA, 55 bp, Artificial Sequence; identical to SEQ ID NO: 40
  noApt (no aptamer control)

SEQ ID NO: 49 - DNA, 3558 bp, Artificial Sequence
  SEAP-2A-eGFP
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcctaggcgtttagtgaaccgtcagaatcgatcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggatatcgattgatggctgtaagcttggaccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtataagggaagcggagctactaacttcagcctgctgaagcaggctggagacgtggaggagaaccctggacctatggttctggggccctgcatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagcagccaggatcctaaaagggcagaagaaggacaaactggggcctgagatacccctggctatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgatgtgatcctgggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggcctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccacggtcatcacgaaagcagggcttaccgggcactgactgagacgatcatgttcgacgacgccattgagagggcgggccagctcaccagcgaggaggacacgctgagcctcgtcactgccgaccactcccacgttttctccttcggaggctaccccctgcgagggagctccatcttcgggctggcccctggcaaggcacgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacgcacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccagggcggtcccggtccaagcgtctggattgagaattccctttcggggcagacatgataagatacattgatgagtttggacaaaccacaactagaatgcagtgaaaaaaatgctttatttgtgaaatttgtgatgctattgctttatttgtaaccattataagctgcaataaacaagtt

SEQ ID NO: 50 - DNA, 154 bp, Artificial Sequence
  hTel - H4_Gq
ctgctcacctgccagctacggacgcggccacgtttagggttagggttagggttagggttcgtggcagcgcgttggtggggttcccgggagggcggctacgggttccgtaatcagatttgtgtacgcgccgcgtccgtagctggcaggtgagcag

SEQ ID NO: 51 - DNA, 174 bp, Artificial Sequence
  3x nucl
ctgctcacctgccagctacggacgcggccacgtggtggtggtggttgtggtggtggtgggcgtggcagcgcgttggtggtggtggttgtggtggtggtgggacgcgcagcgagctggtggtggtggttgtggtggtggtggggctcgccgcgtccgtagctggcaggtgagcag

SEQ ID NO: 52 - DNA, 155 bp, Artificial Sequence
  H4_Gq - nucl
ctgctcacctgccagctacggacgcggccacgtggtggggttcccgggagggcggctacgggttccgtaatcagatttgtgtcgtggcagcgcgttggtggtggtggttgtggtggtggtgggacgcgccgcgtccgtagctggcaggtgagcag

SEQ ID NO: 53 - DNA, 169 bp, Artificial Sequence
  4xEpCAM
ctgctcacctgccagctacggacgcggccacgacagaggttgcgtctgtcgtggcgcgcgtacagaggttgcgtctgtacgcgcagcgagcacagaggttgcgtctgtgctcgccgcccaacagaggttgcgtctgttgggcgcgcgtccgtagctggcaggtgagcag

(19) United States
(12) Patent Application Publication
(10) Pub. No.: US 2023/0357790 A1
(43) Pub. Date: Nov. 9, 2023

(54) SELF-TARGETING EXPRESSION VECTOR

(71) Applicant: Touchlight IP Limited, Hampton (GB)

(72) Inventors: Paul James ROTHWELL, Hampton (GB); Michal LEGIEWICZ, Hampton (GB); Thomas Antony James ADIE, Hampton (GB); Leonor Maximo QUINTANEIRO, Hampton (GB)

(21) Appl. No.: 18/245,656

(22) PCT Filed: Sep. 20, 2021
(86) PCT No.: PCT/GB2021/052439

§ 371 Date: Mar. 16, 2023

(30) Foreign Application Priority Data
Sep. 18, 2020 (GB) 2014751.8

(51) Int. Cl. C12N 15/85; C12N 15/64; C12N 15/115

(52) U.S. Cl. CPC C12N 15/85; C12N 15/64; C12N 15/115; C12N 2310/16

(57) ABSTRACT

The present invention concerns new nucleic acid molecules which may be used in many applications, and methods for making the same. These nucleic acid molecules are preferably DNA vectors, optionally DNA expression vectors. The nucleic acid molecules are able to target the vector to a specific cellular location, such as the nucleus, due to the presence of one or more particular binding motifs within the nucleic acid molecule itself. Thus, the nucleic acid molecules of the invention may also be described as targeted delivery vectors, notably self-targeted delivery vectors or smart delivery vectors.

### FIELD OF THE INVENTION

The present invention concerns new nucleic acid molecules which may be used in many applications, and methods for making the same. These nucleic acid molecules are preferably DNA vectors, optionally DNA expression vectors. The nucleic acid molecules are able to target the vector to a specific cellular location, such as the nucleus, due to the presence of one or more particular binding motifs within the nucleic acid molecule itself. Thus, the nucleic acid molecules of the invention may also be described as targeted delivery vectors, notably self-targeted delivery vectors or smart delivery vectors. Therefore, these constructs can be described as targeting, as they are capable of effectively delivering a payload to a location where expression is desirable. The present invention also relates to a unique method of making vectors. The vector of the present invention may be closed, i.e. without any free ends, or it may be open, wherein the terminal nucleotides are base paired within said vector but not linked to each other. Effectively disclosed is a vector with at least one “sticky end” that permits the vector to be targeted to a desired location.

### BACKGROUND TO THE INVENTION

It is a goal to introduce genetic material into cells, for numerous reasons including in order to compensate for, or correct anomalous genes or to make a beneficial protein. If a mutated gene causes an absence in or mutation of an essential protein, the introduction of a normal copy of the gene to restore cellular function is desirable. Further, the introduction of genetic material encoding an active RNA entity rather than a peptide is desirable.

The genetic material such as a gene is generally not directly applied in therapy. It is included within a vector for delivery to the cell. The delivery of genetic material into cells can be accomplished by multiple methods. Certain viruses are often used as vectors because they can deliver genetic material by infecting the cell. Naked DNA or DNA complexes (non-viral vectors) are also used.

Non-viral vectors present certain advantages over viral vectors, such as larger scale production and low host immunogenicity. However, non-viral vectors such as plasmids may produce lower levels of transfection and expression, and thus lower efficacy. Traditional non-viral vectors such as plasmids may also be amplified in bacteria, meaning that there is the possibility of contamination of the genetic material, as discussed in more detail below.

Non-viral vectors such as plasmids and mini-circles may face major delivery hurdles, notably how to get the nucleic acid to the correct cells, getting it into the cell, and further getting it into the nucleus or other intracellular location, as required. Many barriers exist for the efficient transfer of genetic material to cells, including the extracellular matrix, the endosomal/lysosomal environment, the endosomal membrane, and the nuclear envelope.

Nuclear import is a very well-known bottleneck for gene expression in eukaryotic cells, and a relatively small fraction of transfected DNA is translocated to the nucleus.

Various strategies have been employed, including tagging non-viral vectors with peptides such as a nuclear localization signal or sequence (NLS). Utilisation of duplexed DNA nuclear targeting sequences (DTS) have also increased the ability of vectors to localise to the nucleus but these are included within a duplex and are not binding motifs as described herein. Further, DNA has been complexed with liposomes with surface functionalisation in order to attempt targeting. The use of liposomes is not without limitation, since it is not possible to permit systemic delivery due to the toxicity of cationic lipids.

A targeted delivery system normally includes the DNA of interest, a polycation, usually polylysine or cationic lipid, and a targeting ligand, which is conjugated to the polycation. However, there is a possibility that the DNA may dissociate from the targeting ligand during circulation. Furthermore, there is a need to conjugate a functional domain directly to DNA while maintaining the activity of the DNA. For example, when a NLS was conjugated with plasmids, it has been demonstrated a significant increase of nuclear uptake of plasmids occurred. However, due to the conjugation method used, the plasmids lost their expression activity after conjugation (Sebestyen et al, Natl. Biotech., 16 (1998), pp. 80-85). Thus, conjugation can result in a decrease in the expression from the DNA.

Additional provision of chemically targeted delivery is a hallmark of DNA vectors that include virally derived ITRs. For example, the capsid-free AAV vector described in WO2019/143885 includes virally derived sequences, such as the ITR sequences, are delivered using a lipid nanoparticle delivery system described in WO2019/051289. Analogously, WO2019/246544 describes vectors with virally derived ITRs that relies on secondary agents to target the DNA vector.

Without targeting, a far greater amount of vector is required to ensure the desired effect since much is lost before reaching its desired destination. It is therefore desirable to be able to reduce the amount of vector provided.

However, the tagging of DNA with peptides and indeed proteins and other small molecules can be challenging and means that the vector includes additional entities. It would be far more desirable to offer an “all in one” or minimal solution which permits specific targeting whilst not requiring additional entities to be tagged on the vector for targeting. A single unit for targeted delivery is therefore appealing.

Typically used nucleic acid molecules in the art, such as gene delivery vectors derived from viral genomes may be problematic as they can induce an immune response in the recipient of the gene delivery vector, since the immune system can recognise the circulating “foreign” DNA. Such gene delivery vectors may have viral sequences such as inverted terminal repeats (ITRs), which may provoke the innate immune system and also recruit DNA repair enzymes which inhibit expression from the vector. If DNA is produced in bacterial cells, it will have prokaryotic patterns of DNA methylation which may be identified as foreign within eukaryotic organisms, and similarly rejected. For example, plasmids (pDNA) are circular dsDNA molecules which are naturally occurring, extra chromosomal DNA fragments stably inherited from one generation to the next. Plasmids and derivatives thereof have been used as gene delivery vectors with varied amounts of success.

The method of producing the nucleic acid vectors may also be problematic. Manufacturing nucleic acid structure within bacterial cells risks the contamination of the final product with lipopolysaccharides (LPS), endotoxins and other prokaryotic-specific molecules. These have the capability to raise an immune response in eukaryotic organisms, since they are effectively an indicator of a microbial pathogen. Indeed, manufacturing nucleic acid vectors within any cell-based system results in the risk of contaminants from the cell culture being present within the final product, including genomic materials from the host cells. Production of nucleic acids within cells is inefficient, since many more materials are required to be supplied to produce the nucleic acid than a synthetic method. In addition to the issues of cost, use of cell cultures can in many cases present difficulties for reproducibility of the amplification process. In the complex biochemical environment of the cell, it is difficult to control the quality and yields of the desired nucleic acid product. It is also difficult to deal with sequences that may be toxic to the cells in which the nucleic acid is amplified. Recombination events may also lead to problems in faithful production of a nucleic acid of interest.

DNA may be produced synthetically without the use of cells. Oligonucleotides may be synthesised chemically by extension of a chain using modified nucleotides. Preparation of these building blocks comes with a cost. The stepwise addition of each nucleotide is an imperfect process (the chance of each chain being extended is termed the ‘coupling efficiency’), and for longer sequences a majority of the initiated chains will not become full-length correct products. This precludes production of long sequences at large scale—there must always be a sacrifice between length, accuracy, and scale for these processes. Primary uses for such oligonucleotides are still in the low hundreds of nucleotide range (for example, primers and probes), and the maximum accurate length is thought to be around 300 nucleotides in length. Typically, synthetic oligonucleotides are single-stranded nucleic acid molecules around 15-25 bases in length.

A preferred alternative to synthetic processes is the enzymatic production of nucleic acids, which relies upon a template. Cell-free, in vitro enzymatic processes for the synthesis of nucleic acid avoids the requirement for use of any host cell, and so are advantageous, particularly when production is required to Good Manufacturing Practices (GMP) standards. Consequently, enzymatically produced nucleic acids can be made much more efficiently, and without the risk of cell-derived contaminants.

Therefore enzymatically produced and improved constructs which are safer and more tolerable by the recipient are required, ideally that are also resistant to immediate degradation within the cell. Further, it is desirable if these improved constructs are targeting, for example that they are capable of directing the construct towards a particular tissue or cell type, or a particular location within a cell, including the nucleus. Targeting a vector towards a desired location is a goal of gene therapy and the like. Alternatively, targeting a particular bacterial cell can permit the expression of a bactericidal agent within a particular cell type. Similar approaches may be taken with other microorganisms such as fungi or protists.

The present invention relates particularly to a novel, cell-free and in vitro method for making targeting nucleic acid constructs efficiently and effectively, and also to the targeting constructs themselves.

The available art does not disclose synthetic targeting constructs/vectors made substantially from DNA, nor does it describe a method for manufacturing such constructs.

### BRIEF DESCRIPTION OF FIGURES

FIG. 1: Graph showing the comparison of the performance of vectors that include different structural motifs and hence binding motifs in the cap on the right side of the vector. The binding motifs were chosen to interact with target proteins to hijack nuclear import pathways and enhance nuclear import of the reported DNA; tested in HEK293. In this Example the vector was completely covalently closed.

FIG. 2: Graph showing the performance of vectors with a single binding motif within the cap transfected at moderate concentration; tested in HEK293.

FIG. 3: Graph showing the performance of vectors with a single binding motif within the cap transfected at low concentration; tested in HEK293.

FIG. 4: Graph showing the performance of vectors with a single binding motif within the cap transfected at moderate concentration; tested in HepG2.

FIG. 5: Depicts various embodiments of the vector of the present invention wherein the vectors are all covalently closed. At the top is shown a vector with G-quadruplexes at both the left and right ends. The second vector includes an aptamer at the left end and a different aptamer at the right end. The third structure includes a G-quadruplex at one end and an aptamer at the other. The fourth structure includes a stem loop at each end, wherein the loop is a single strand of DNA, held together by a stem structure. In this case the loop can be a simple trinucleotide such as GAA.

FIG. 6: depicts various embodiments of the invention wherein the vector is not covalently closed and includes one or more nicks. All examples include a G-quadruplex at the left and right ends. The nick can be present in the duplex at either end of the duplex, or at both ends of the duplex as depicted.

FIG. 7A: Depicts exemplary vectors used in the Examples of the present application. The top is a simplified depiction of the vector, which includes a cap at one end which includes multiple streptavidin aptamers, whilst the other is a simple stem loop (GAA in the loop). Also shown is the expression cassette present in the vector, and the vector that included a single aptamer. FIG. 7B depicts part of the experimental protocol used in Example 4.

FIG. 8: This is a graph of the binding experiment shown in FIG. 7B. Shown is the improved ability of the vector which includes 4 streptavidin aptamers to bind, whilst good binding results are also obtained for a single aptamer. The control (no aptamer) shows no specific binding.

FIG. 9: This depicts the various caps used in the Examples on the vector. The various capped ends used are: a stem loop, an aptamer, a G-quadruplex and a multiplex-aptamer.

FIG. 10: Depicts one embodiment of the method described herein. Shown is a double stranded template molecule (1) which includes the sequence encoding the processing motifs and the structural motifs. Once in single stranded form, the processing motifs form and can be processed using an appropriate enzyme such as an endonuclease (scissors and dotted line) (2). Once the processing is complete (3), the molecule can be contacted with a polymerase to fill-in the duplex which can be left open (4) or covalently closed with a ligase (5).

FIG. 11: Is a depiction of a template used in Example 5.

FIG. 12: Is a gel photographs showing the results of using the method of the present invention to generate targeting vectors, as detailed in Example 5.

FIG. 13: Graph showing the performance of vectors with multiple binding motifs within a single cap transfected at moderate concentration; tested in HEK293.

### SUMMARY OF THE INVENTION

The present invention relates to a targeting vector, herein also called a construct interchangeably throughout. The targeting vector may also be termed a targeting delivery vector. The targeting vector is preferably a DNA vector or a hybrid of DNA and RNA. The vector includes a portion of duplexed nucleic acid. The targeting vector is preferably an expression vector. The genetic material for “expression” may be included in the duplex. The duplexed section is capped at each end. These caps may be entirely continuous with the duplex, i.e. the capped end is a closed end, or may be continuous with one strand of the duplex only and include a nick or larger gap between the terminal nucleotides of the “non-continuous” strand. The vector may be comprised of a single strand of nucleic acid, which is continuous (covalently closed) or discontinuous (includes nicks or gaps). In some embodiments, there is a single gap or only a nick present in one capped end. The vector includes at least one structural motif in at least one capped end. The structural motif includes at least one binding motif that is capable of targeting, guiding or directing the vector. The vector is preferably synthetic and made in a cell-free manner enzymatically.

The target for the vector is preferably cellular. A cellular target is one associated with a cell. The target may be any appropriate entity upon or within the cell to enable targeting of that cell or cell location (i.e. nucleus). Thus, targeting as used herein is to enable nucleic acids to be delivered to a target cell in preference to non-target cells or target cellular locations to assist expression levels.

Accordingly, there is provided a targeting DNA vector including a duplexed section characterised in that the duplex is capped at both ends, wherein at least one end of the duplex is capped with a structural motif and said structural motif includes at least one binding motif.

The targeting DNA vector may be a delivery vector. The vector may be delivered to a desired target cell or a desired target location in a cell.

Independently, the vector may comprise any one or more of the following optional features:

The duplex DNA may be capped at both ends with a structural motif, which can be the same or different.

Each of the capped ends may be independently covalently closed, i.e. continuous with the duplexed DNA, or be open, i.e. include a nick or gap.

The duplex DNA may be independently capped at one end with a structural motif and at the second end with a hairpin, a T shaped hairpin, a stem loop, a loop, a bulge or a cruciform.

The structural motif may include a plurality of binding motifs, which can be the same or different. The structural motif can include and array of binding motifs, such as 3 or more binding motifs, which can be the same or different. Thus the array may be a multiplex of binding motifs.

The binding motif is responsible for the targeted delivery of the vector. The binding motif binds to the cellular target. The binding motif forms a conformation which permits binding to the cellular target.

The binding motif is capable of binding to a target on any one or more of:
(i) a cell surface;
(ii) the nuclear envelope;
(iii) the nuclear transport system;
(iv) a cellular compartment;
(v) a nuclear component;
(vi) a cytoplasmic inclusion; and/or
(vii) a cytoplasmic protein or peptide

It may be preferred that the binding motif enables nuclear targeting. Thus, the binding motif may be specific for entities commonly transported to the nucleus of the cell. Such entities include histones, nucleolin, telomere binding proteins and the like.

It may be preferred that the binding motif is designed such that known sites that recruit DNA repair enzymes are not included. Representative sites include viral ITRs.

It may be preferred that the binding motif is designed such that it is the conformation or the combination of conformation and key/specific residues and/or sequence that is responsible for the binding specificity of the binding motif.

For the avoidance of doubt, a binding motif as used herein is not a consensus sequence present in double stranded DNA. Representative consensus sequence sites are restriction endonuclease sites, methyltransferases and transcription factors. Such do not require a particular conformation. Binding is determined by structure rather than primary sequence.

Optionally, the binding motif is not derived from virally derived genomic sequences, such as inverted terminal repeats and the like.

Notably, it is presented here that the binding motifs are within the structural motif, and are effectively provided as a single strand. If the binding motifs are present with a complementary sequence also present, this may lead to potential double strand interference. In this scenario, the formation of a duplex or double stranded sequence would compete with the formation of the conformation of the binding motif, which is undesirable. If the binding motifs were present with a complementary sequence, each binding motifs would be present as a sense and an antisense version, and as such could form a duplex. Thus, including the binding motifs within the structural motif is preferable, rather than within the duplex section.

The vector may also include a further binding motif capable of binding to any one or more of:
(i) a peptide or protein;
(ii) a small molecule;
(iii) an antibody or derivative thereof;
(iv) an enzyme;
(v) an immunostimulant;
(vi) an agonist or antagonist;
(vii) an adjuvant and/or
(viii) a nucleic acid.

Such a binding motif may be distinct to the binding motif responsible for targeting. Thus, this is a further binding motif provided to the vector to provide additional functionality to the vector.

The targeting vector may be directed to a cellular target which is present in or on a eukaryotic cell, optionally a plant cell, protist cell, fungal cell, human cell or a non-human animal cell. The target entity may be any suitable cellular target.

The targeting vector may be directed to a cellular target which is present on or in a prokaryotic cell, such as a bacterial cell. Optionally said cell is a gram negative or gram-positive bacterial cell. The targeting vector is delivered to the target by virtue of the specific binding of the binding motif.

It may be preferred that the vector is delivered to the cell nucleus by virtue of the binding motif(s). The target may be a histone, a nucleolin, and/or a telomere binding protein.

The duplex DNA may include a gene sequence or a fragment thereof and optionally a promoter. The promoter may be operatively linked to the gene sequence or fragment thereof.

The targeting vector may be a targeting expression vector.

The targeting vector may include modified nucleotides, optionally modified nucleotides in the capped ends.

The targeting vector may be substantially pure DNA, optionally 95% DNA (by weight).

The targeting vector may be a DNA/RNA hybrid.

The structural motif may permit the formation of hydrogen bonds between the nucleotide bases in the sequence of the structural motif, optionally wherein said hydrogen bonds between the nucleotide bases involve Watson-Crick base pairs, Hoogsteen base-pairs or non-canonical base-pairing.

The structural motif forms a non-canonical DNA structure, and may include any one or more of:
a) a hairpin;
b) a cross-arm;
c) a triplex;
d) a G-triplex;
e) a G-quadruplex;
f) an i-motif;
g) a pseudoknot;
h) a stem loop; and/or
i) a bulge or loop.

The structure or conformation the binding motif assumes may permit the association with the target in a structure and/or sequence-dependent manner.

The binding motif may be any one or more of:
a) an aptamer;
b) a G-quadruplex;
c) a catalyst;
d) and i-motif and/or
e) triple stranded DNA.

The binding motif may be specific. By specific it is meant that the binding motif binds selectively to the target entity in preference to any other entity. This is due primarily to the conformation it forms, but can also include the presence of specific or key residues.

The present invention also relates to a method of delivering a DNA vector to a target cellular location, comprising the administration of a vector as described herein to a recipient. The recipient may be a human or animal in need thereof, or may be a cell, tissue or organ in vitro. Thus, the invention extends to use of the vector as described herein for delivery of a DNA vector to a target location.

The present invention also relates to a method of manufacturing a nucleic acid vector. The vector may be as described herein.

The steps of said method may comprise:
(a) provision of a nucleic acid template comprising a sequence encoding:
   (i) a first processing motif, adjacent to
   (ii) a first structural motif comprising at least a portion of a first capped end,
   (iii) a single strand of said duplex DNA,
   (iv) a second structural motif comprising at least a portion of a second capped end, adjacent to
   (v) a second processing motif
said processing motif includes a sequence capable of forming a base-paired section including a recognition site for an endonuclease containing a cleavage site,
said structural motif includes at least one sequence capable of forming intramolecular hydrogen bonds,
either or both of said first or second capped ends include a structural motif containing a binding motif;
(b) amplifying said template using a polymerase capable of rolling circle amplification such that a single stranded concatemer is produced;
(c) contacting the single stranded concatemer with an endonuclease to release single stranded DNA constructs wherein the 3′ terminal nucleotide is base paired adjacent to a single stranded portion of the construct; and
(d) contacting the single stranded DNA construct with a polymerase enzyme to extend the 3′ terminal nucleotide using the single stranded DNA construct as a template to form the duplex section.

The amplification part of this method may require the use of a primer or may be initiated using a primase or a nickase enzyme.

It will be appreciated by those skilled in the art that using the method described here will result in a vector wherein one of the capped ends is nicked or gapped. Thus, one of the capped ends is a closed end. Should a nick be required in both ends, this can be achieved using a nickase enzyme.

The 5′ terminal nucleotide may also be base paired to the single stranded portion of the construct, and the polymerase may construct the duplex as far as the 5′ terminal nucleotide. The nick between the 5′ nucleotide and 3′ nucleotide may then be appropriately ligated.

The method may further comprise step (e) the addition of a suitable enzyme or reagent to covalently close the nick or gap. A suitable enzyme may be a ligase.

The template nucleic acid may include a portion of a capped end which is greater than the whole of the sequence required to form the capped end. In such an instance, the single stranded product transcribed therefrom may be contacted with a nickase prior to extension with a polymerase enzyme. The nickase may expose an appropriate 3′ terminal nucleotide for extension.

### DETAILED DESCRIPTION OF THE FIGURES

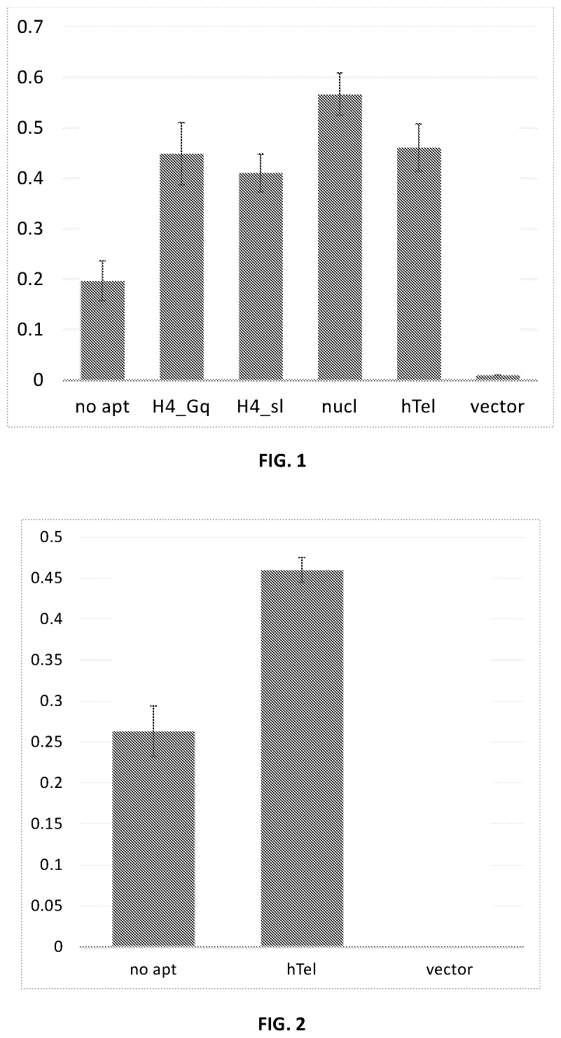

FIG. 1 is a graph showing the comparison of normalized SEAP expression (U/mL of media) from the vector in HEK293 cells that were transfected with different vectors that different in terms of the binding motif selected for use in the cap. The binding motifs were chosen to interact with target proteins that are imported to nucleus:histone H4:H4_Gq, and H4_sl, nucleolin:nucl, and factors recognizing human telomere G-quadruplex: hTel. The graph is a bar chart showing the results (SEAP expression in U/mL) versus vector used.

FIG. 2 is a graph showing normalized SEAP expression (U/mL of media) in HEK293 cells that were transfected with reduced amount of vector containing a single binding motif on the right end. The binding motif was a human telomere G-quadruplex motif. 0.4 μg vector with capped ends was doped with 0.5 μg competitor vector (no SEAP). The graph is a bar chart showing the results (SEAP expression in U/mL) versus vector used.

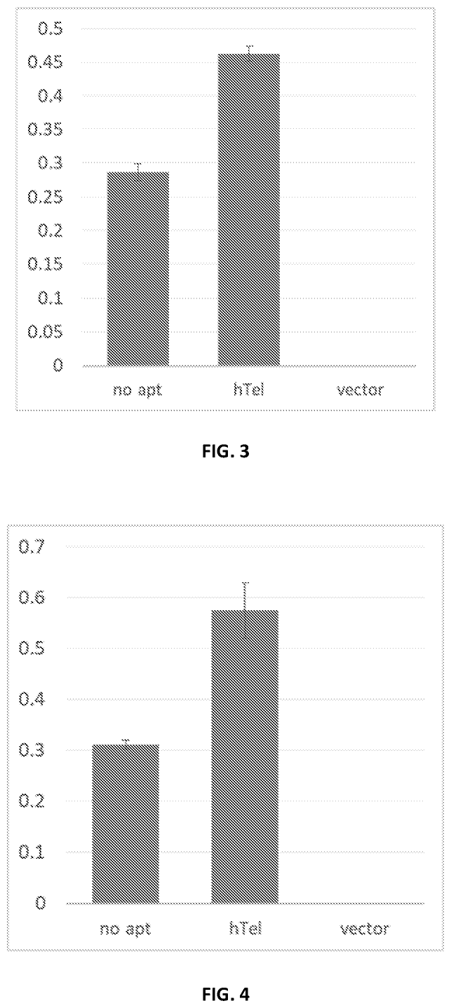

FIG. 3 is a graph depicting normalized SEAP expression (U/mL of media) in HEK293 cells that were transfected with reduced amount of vector containing a single binding motif on the right end. The binding motif was a human telomere G-quadruplex motif. 0.2 μg vector with a binding motif was doped with 0.7 μg competitor vector (no SEAP). The graph is a bar chart showing the results (SEAP expression in U/mL) versus vector used.

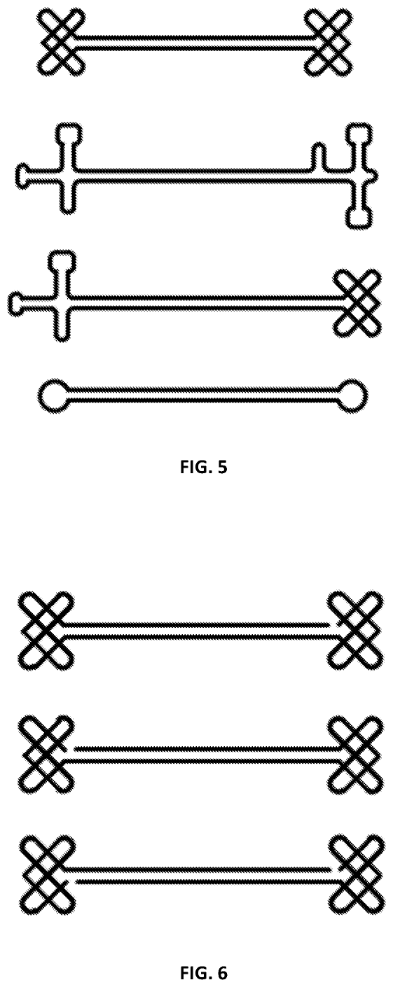

FIG. 4 is a graph depicting SEAP expression (U/ml of media) in HepG2 cells transfected with reduced amount of vector containing a single binding motif on the right hand end. The binding motif was a human telomere G-quadruplex motif. 0.4 μg vector with a binding motif, 0.5 μg competitor vector (no SEAP) was used. The graph is a bar chart showing the results (SEAP expression in U/mL) versus vector used;

FIG. 5: Depicts various embodiments of the vector of the present invention wherein the vectors are all covalently closed. At the top is shown a vector with G-quadruplexes at both the left and right ends. The second vector includes an aptamer at the left end and a different aptamer at the right end. The third structure includes a G-quadruplex at one end and an aptamer at the other. The fourth structure includes a stem loop at each end, wherein the loop is a single strand of DNA, held together by a stem structure. All include a duplexed section of DNA and at least one capped end formed from a structural motif and including a binding motif such that the vector can be targeted or directed as desired.

FIG. 6: depicts various embodiments of the invention wherein the vector is not covalently closed and includes one or more nicks or gaps. All examples show the linear duplex section and also the capped ends. All exemplary vectors shown here include a G-quadruplex at the left and right ends. The nick (simple backbone break) or gap (one or more nucleotides of single strand in the otherwise duplexed section) can be present in the duplex at either end (right or left) of the duplex, or at both (right and left) ends of the duplex as depicted.

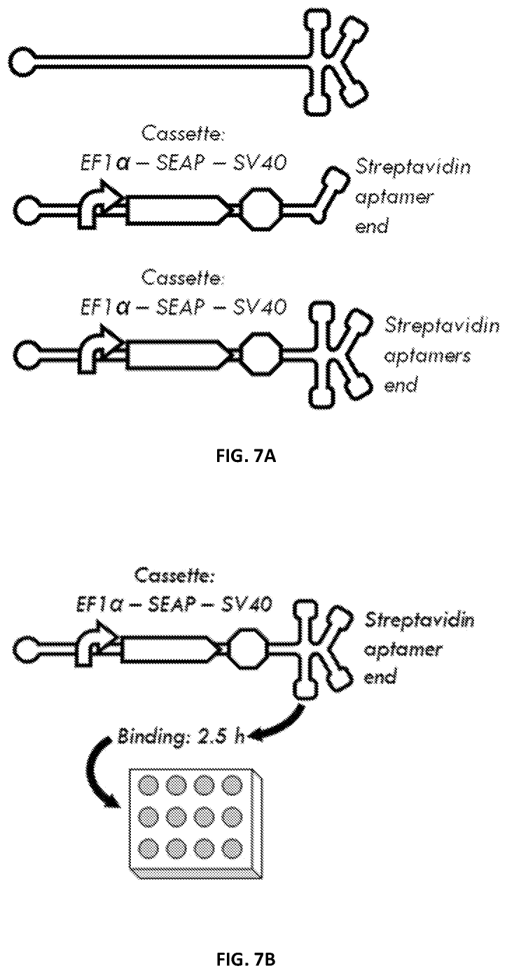

FIG. 7A: Depicts exemplary vectors used in the Examples of the present application. The top vector is a simplified depiction of the vector, which is a duplexed linear vector with a cap at one end which includes multiple streptavidin aptamers, whilst the other cap is a simple stem loop (GAA in the loop). The vector in this instance is covalently closed. Also shown is the expression cassette present in the vector, with promoter (EF1α), gene (SEAP) and polyA signal (SV40). In this instance the vector includes a capped end with a single aptamer as a binding motif, this aptamer binding to streptavidin, or a capped end that includes 4 streptavidin aptamers. FIG. 7B depicts part of the experimental protocol used in the Examples, where the vector is exposed to streptavidin-coated plates for two and a half hours before the unbound vector is washed away. A vector without the aptamer is used as the control. An intercalating fluorophore is then use to bind to the vectors bound to the plates for detection purposes.

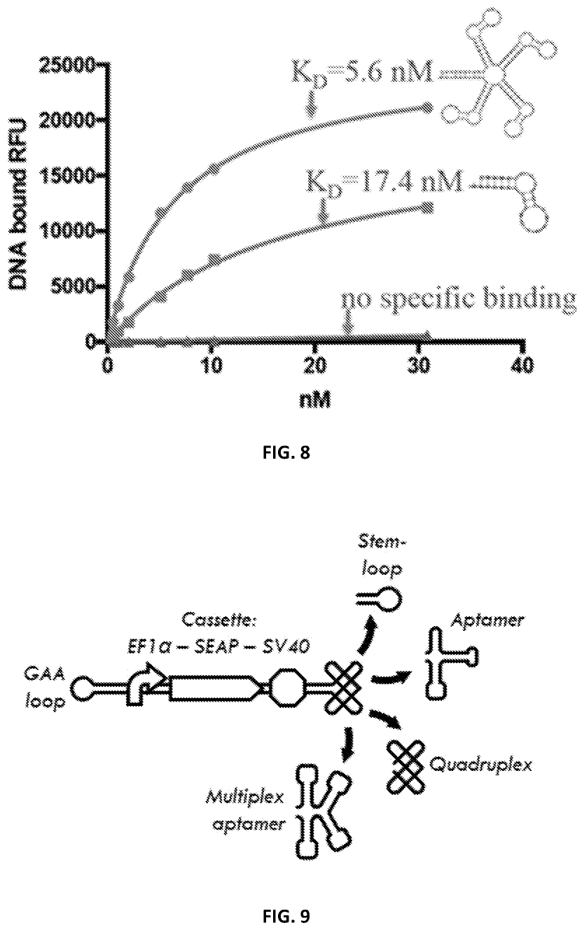

FIG. 8: This is a graph of the binding experiment shown in FIG. 7B, using three vectors. These vectors are a control (no aptamer), a single aptamer, and a capped end including four aptamers. Shown is the improved ability of the vector which includes four streptavidin aptamers to bind, whilst good binding results are also obtained for a single aptamer. The control (no aptamer) shows no specific binding and is washed away. The graph is a plot of concentration of vector in nM versus DNA bound in RFU (relative fluorescence units).

FIG. 9: This depicts the various alternative capped ends employed for the vectors made and tested in the Examples. The various capped ends used are: a stem loop, an aptamer, a G-quadruplex and a multiplex-aptamer. In this instance all of the capped ends have been included in the right end of the vector (as viewed in terms of the sense sequence for the gene) but those skilled in the art will appreciate that either “end” (left or right) can include the binding motif.

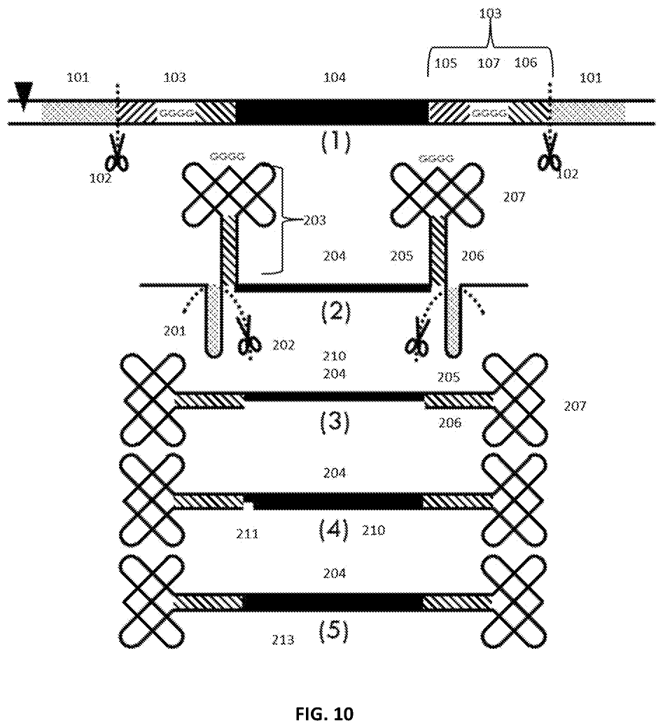

FIG. 10: Depicts one embodiment of the method described herein. Shown is a double stranded template molecule (1). The triangle indicates a suitable nicking site, which enables the double stranded template to be nicked and therefore initiate amplification of only one strand of the template). Shown are the sequences encoding the processing motifs (101) and the structural motifs (103). In this case the structural motif includes 3 components two sequence: Two sequences to form a structural stem (105 and 106) and a central sequence that forms the binding motif (107). Once in single stranded form (2), the processing motifs (201) form and can be processed using an appropriate enzyme such as an endonuclease (202). It can be seen that the structural motif (203) has permitted a structure to form, which in this instance is a stem formed between (205) and (206) with a G-quadruplex (207) intervening. Following processing (3), the molecule can be contacted with a polymerase to fill-in the duplex (210) which as shown in (4) can be left open (a nick (211) is shown) or as shown in part (5) covalently closed via ligation (213). To assist clarity, only one end has been labelled at a time, but the labels can equally apply to the other end.

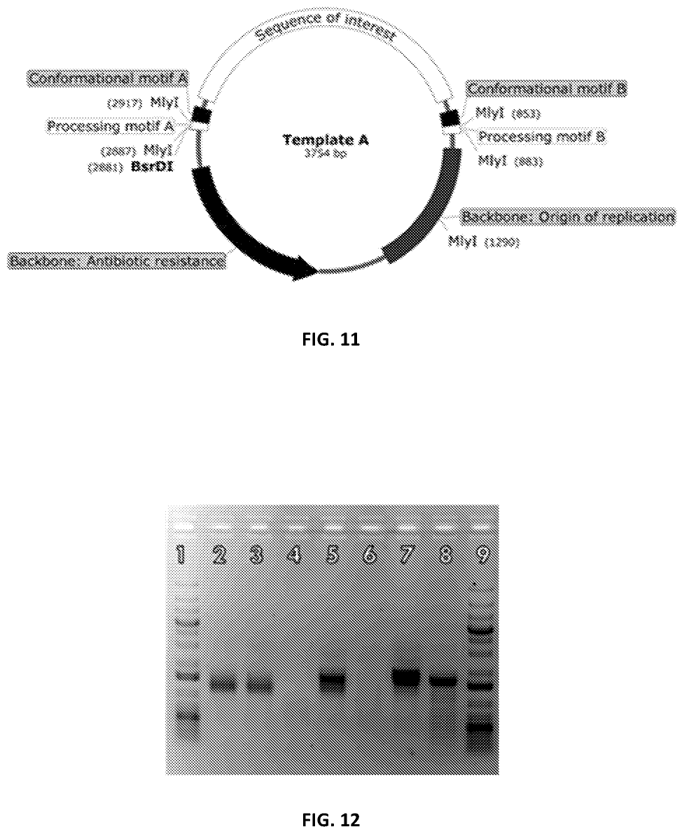

FIG. 11 is a depiction of the template used in Example 5 includes a nicking site, a processing motif adjacent to a conformational motif, a sequence of interest, a second conformational motif adjacent to a second processing motif, and a backbone of similar size to the sequence of interest. There is an additional endonuclease target site in the backbone, which will only cut in dsDNA.

FIG. 12 shows a 0.8% agarose gel stained with SafeView demonstrating production of vectors in Example 5 by second-strand synthesis and ligation. Lanes 1 and 9 are Thermo Scientific Gene Ruler 1 kb Plus DNA ladder. Lane 2 lacks all enzymes; lane 3 includes T4 DNA ligase; lane 4 includes T4 DNA ligase and the T5 exonuclease clean-up step; lane 5 includes T4 DNA polymerase; lane 6 includes T4 DNA polymerase and the T5 exonuclease clean-up step; lane 7 includes both T4 polymerase and T4 ligase; lane 8 includes both T4 polymerase and T4 ligase and the T5 exonuclease step.

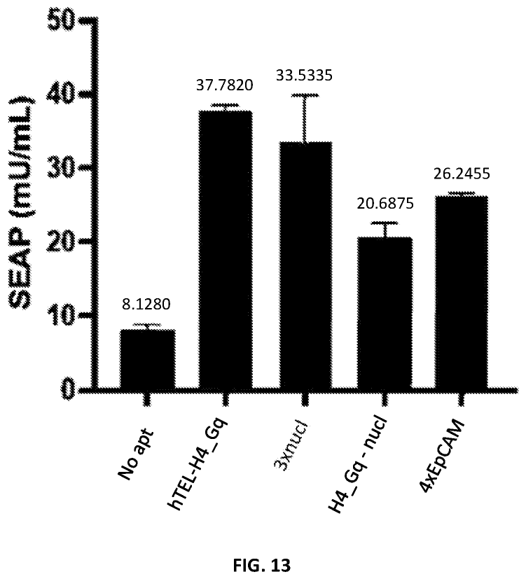

FIG. 13 shows a graph depicting normalized SEAP expression (U/ml of media) in HEK293 cells that were transfected with reporter DNA containing multiple structured terminal motifs on a single end, as described in Example 6.

### DETAILED DESCRIPTION OF THE INVENTION

The present invention meets the need of the provision of a vector which may target and deliver itself to a desired cellular location. Thus, the vector may be described as targeting, since it requires no additional assistance to reach its desired location. It is envisioned that the desired location may be a tissue type, cell type, and/or location within a cell. The desired location may be a particular strain of pathogen, such as a bacteria or fungi. Said cell may be in vivo or ex vivo or in vitro. The vector is capable of targeting due to the presence of binding motifs included within the vector, notably within the structural motif. It is preferred that there is one or more binding motifs included. It is preferred that the binding motifs are present in the capped ends, rather than the duplex or linear section. Experimentally, the present inventors have demonstrated that the inclusion of a plurality of binding motifs permits even further improved targeting. The vector may be described as including a section of duplexed nucleic acid, which is capped at both ends. The duplex may be described as a linear section. The caps may be the same or different. The caps may be open ended or closed. At least one cap includes a structural motif. Said structural motif is capable of assuming a structure. The structural motif includes the one or more binding motifs. The binding motifs together may form the structural motif, or the structural motif can support the structure of one or more binding motifs. The vector may have identical capped ends, or each end may be different. Only one end is required to include binding motifs for targeting. The other end may include binding motifs for other functions such as transporting a small molecule. The vector, therefore, may be asymmetric.

The vector is designed such that it is delivered by virtue of the specific binding via the binding motif(s) to a desired cellular target. The vector is designed such that sequences and structures known to recruit DNA repair enzymes are avoided, since the DNA damage repair pathway initiation can reduce expression from a vector if this is desired. Such structures may include viral ITRs which are preferably excluded.

Vector

The present invention relates to a nucleic acid vector, preferably a DNA vector. A nucleic acid vector can be defined as a vehicle to carry genetic material into a cell, where it can be replicated and/or expressed. The purpose of a vector which transfers genetic information to cell is typically to isolate, multiply, or express the insert in the target cell. Vectors may be designed for transcription into RNA and/or protein expression. Vectors designed specifically for the expression of a transgene or fragment thereof in a target cell may have a promoter sequence that drives expression of the gene or fragment thereof. The vector according to the present invention can be any suitable type of vector and can result in the expression of any type of RNA or protein within the cell. The vector permits the translation of information encoded in a gene or fragment thereof into protein or RNA structures in the cell. Expressed genes include genes that are transcribed into messenger RNA (mRNA) and then translated into protein, as well as genes that are transcribed into RNA, such as transfer and ribosomal RNAs, but not translated into protein. It may be preferred that the vector is an expression vector. The expression vector may include a gene or fragment thereof, the gene may be a transgene, i.e. a gene that is not already present in the cell into which it is introduced. The gene may encode a protein or an RNA entity.

Expression vectors may produce proteins intracellularly through the transcription of the gene or fragment thereof followed by translation of the mRNA produced. Expression in different organisms results in differing requirements for enabling the production of protein, although many of the elements are similar. In general, the elements required may be a promoter for initiation of transcription, and a termination signal. Included may be an expression cassette. For expression in eukaryotic cells, the expression cassette comprising one or more promoter or enhancer elements and a gene, a fragment of a gene, or other coding sequence which encodes an mRNA or protein of interest. The expression cassette may consist of a eukaryotic promoter operably linked to a sequence encoding a protein of interest, and optionally an enhancer and/or a eukaryotic transcription termination sequence. Examples of genes or coding sequences of interest for a eukaryotic system may include the coding sequence for an antigenic entity, and therefore the vector may be a nucleic acid vaccine. For a prokaryotic expression vector, the vector may include a prokaryotic promoter and a termination sequence. Promoters in any expression vector or expression cassette may be inducible, meaning that expression is only initiated when required by the introduction of an inducer.

Expression vectors designed to produce RNA without the production of protein may include a suitable promoter to drive transcription of the gene or fragment thereof. The gene or fragment thereof may encode any suitable RNA molecule, for example a messenger RNA (mRNA), transfer RNA (tRNA), ribosomal RNA (rRNA), antisense RNA (asRNA), guide RNA (gRNA), small interfering RNA (siRNA), microRNA (miRNA), long non-coding RNA (lncRNA), Piwi interacting RNA (piRNA) and short hairpin RNA (siRNA). Alternatively, the RNA could form a ribozyme or aptamer.

Since it is preferred that the vector is artificially synthesized, such as made enzymatically in a cell-free process, it is possible to design any gene or fragment thereof for expression. It may therefore be cytotoxic, which gene sequence is difficult to propagate in bacteria and the like.

The gene, or fragment thereof, therefore encodes for an RNA or protein product within the cell. A gene fragment may relate to pieces of genes containing only the exons (those parts of the gene which actually encode the protein sequence). Alternatively, the gene fragments may encode a monomer or subunit of a larger protein, rather than the entire protein itself. Gene fragments may also be relevant in the production of nucleic acid vaccines, for example the inclusion of a portion of an antigenic protein from a virus for expression in the cell. In such circumstances it may be undesirable to produce the whole protein in the cell. A fragment can therefore be a small piece of a gene if this is appropriate for expression in the cell. Exemplary is a portion of the spike protein of a virus for inducing an immune response.

The vector is preferably a DNA (deoxyribonucleic acid) vector. The vector may be a hybrid vector, meaning that different types of nucleotides are incorporated into the vector, which is possible using the synthesis methods discussed herein. In hybrid vectors, it may be preferred that the duplex section is DNA whilst the capped ends may be another type of nucleic acid such as RNA (ribonucleic acid) or using modified nucleotides. Alternatively, sections of the duplex may be RNA or modified nucleotides. The vector may be 80% DNA or more, 85, 90 or 95% DNA.

It is preferred that the vector is substantially pure nucleic acid, for example that the vector is at least 95% nucleic acid. The vector may be 95, 96, 97, 98, 99 or 100% nucleic acid. Optionally, the vector is substantially pure DNA, for example, the vector is at least 95% DNA. The vector may be 95, 96, 97, 98, 99 or 100% DNA. It may be preferred that the vector is substantially free or protein or peptide, such that less than 5% of the vector is protein or peptide. Optionally, the vector is less than 4, 3, 2, or 1% peptide or protein. It is preferred that the vector does not include a peptide targeting sequence such as NLS. As used herein the percentage refers to percentage by weight of the vector per se. It will be appreciated that using attachment sites, molecules may be attached as therapeutics to the vector to be carried and as such are not material of the vector nor the targeting mechanism.

The vector may be a natural nucleic acid molecule such as DNA or RNA. It is preferred that the vector is DNA. The vector can also include a non-natural nucleic acid molecule. Examples of non-natural nucleic acid molecules or xeno nucleic acids (XNA) include 1,5-anhydrohexitol nucleic acid (HNA), cyclohexene nucleic acid (CeNA), threose nucleic acid (TNA), glycol nucleic acid (GNA), locked nucleic acid (LNA), peptide nucleic acid (PNA) and FANA. Hachimoji DNA is a synthetic nucleic acid analogue that uses four synthetic nucleotides in addition to the four/five present in the natural nucleic acids, DNA and RNA. Enzymes have been engineered, mutated or developed in order to recognise synthetic nucleic acid molecules, and therefore the methods and products of the invention apply equally to these analogues, or hybrids of synthetic and natural nucleic acids and chimeras thereof.

The vector has been depicted in several Figures, including FIGS. 5 and 6. When drawing DNA vectors it is convention to place the “upper strand” of the linear duplex as the “sense strand” in the correct orientation (which would be the 5′-3′ orientation if the ends were open). Such convention carried through the figures included here. For clarity, the capped ends of the vector may be designated “left” and “right” when depicted according to such a convention, rather than 5′ and 3′ which implies there is a free nucleotide at the end, when the molecule may actually be covalently closed and not have any terminal nucleotides. The structural motif may be present at either the left and/or right end.

The vector is a synthetic DNA structure, which is capable of being produced in vitro enzymatically, in a cell-free manner. This synthetic DNA is capable of de novo manufacture, and is not produced by editing natural genomic structures such as chromosomes. The vector does not include centromeric sequences or sequences that act as a centromere. The vector is not an artificial human chromosome.

The vector may be any suitable size, optionally less than 5 Mb in size, such as any size between 0.1 KB and 5 Mb, such as up to 1 Mb, up to 2 Mb, up to 3 Mb, up to 4 Mb or up to 5 Mb. For delivery of genetic sequences, the vector may be any suitable size between 0.1 Kb and 1 Mb, optionally 0.1 Kb and 0.75 Mb, optionally 0.1 Kb and 0.5 Mb, optionally 0.1 Kb and 0.25 Mb (250 Kb). The minimal size will depend on the length of the duplex sequence, but may be in the order of 0.1 Kb, 0.2 Kb, 0.3 Kb, 0.4 Kb or 0.5 Kb. Any intermediate size range between these are possible.

Duplex

The vector may comprise a duplexed section or “duplex”. The duplexed section may also be described as a linear section. A duplex is a section of the molecule having complementary polynucleotide strands, either of DNA or a hybrid of DNA and RNA. It will be understood by those skilled in the art that the duplex can be formed of two complementary polynucleotide strands (intermolecular) or two complementary sections of the same polynucleotide strand (intramolecular). The duplex of the present invention may be of any suitable length. The duplex may be formed by two strands or one strand. The duplex may be pure DNA or a hybrid with RNA. The methods described herein permit the production of a duplex with RNA and DNA.

It is preferred that the gene or fragment thereof is included within the duplexed section. Thus, the entire expression cassette may be present in the duplex, including a promoter and optionally a termination sequence. The promoter may be operably linked to the gene or fragment thereof.

Since the vector may be made artificially there is no requirement to include extraneous sequences such as markers for selection. Such are required when vectors are propagated in cellular environments, such as antibiotic resistance genes.

The duplexed section does not have blunt, open ends, and as such does not have parallel terminal nucleotide residues—the 3′ and 5′ ends of each complementary strand (which can be one strand with self-complementarity). At both ends at least one of the duplexed strands extends beyond the other strand, such that there is an overhang. This overhang may form the capped end. The overhang may continue and connect to the opposite strand of the duplex (closed end).

The duplex may be of any suitable length, and will depend upon the sequence which it is carrying, since some human genes are several kilobases in length. The vector is an effective replacement for plasmids, which can carry an insert of up to 100,000 base pairs. Thus, the duplex section may be up to 100,000 base pairs, 50,000 base pairs or 25,000 base pairs in length.

The binding motifs for targeting and delivery as described herein are preferably not included in the duplex section; they are instead present within the structural motif at the end of the molecule. This is to ensure that the payload of the vector is appropriately delivered without interferences from any structures.

The duplex may contain any appropriate sequence (“payload”) it is desired to deliver to a cell, including a gene, transgene, coding sequence for an active RNA, donor sequence for gene editing and the like.

The duplex may contain appropriate sequences such as promoters, enhancers, termination, polyA signal sequences and the like that increase the activity of the nucleic acid within the target cells. Such are within the remit of those skilled in the art.

Capped End

Each end of the duplex is capped. The capped end or “cap” serves to protect the ends of the duplex, particularly from degradation within the cell via exonucleases. As used herein a capped end of the duplex may assume a structure which may be held together using intramolecular hydrogen bonds. Thus, at least one strand of the duplex continues past the end of the other strand and forms the cap, helping to protect the duplex from degradation in the cell. The duplex is, therefore not blunt ended with a free 5′ and 3′ end. It could be considered that a capped end is formed from one of the duplex strands which is longer than the other, and this single prolonged strand folds into a structure and preferably anneals near the terminal nucleotide of the other strand of the duplex, such as to help to “cap” the end, and sterically prevent the entry of exonucleases and the like. It may be continuous with the other strand in a closed end, therefore providing a covalently closed end.

The capped end may be a closed end, meaning that each end of the duplex is covalently attached to the cap. Alternatively put, the cap and the duplex is a continuous strand.

The capped end may be an open end, meaning that only one end of the duplex is covalently attached to the cap. Alternatively put, only one strand of the duplex continues into the cap, whilst there is a gap between the terminal nucleotide of the cap and the terminal nucleotide of the opposite strand of the duplex. In this situation the terminal nucleotides may be secured within the vector in order to stabilise the vector and prevent immediate degradation within a cell.

One capped end of the duplex may be a simple cap, for example it may be one or more hairpins (continuous) or nicked hairpins (with a gap). Alternatively one end may assume a number of simple conformations, such as a stem loop (a duplex with a loop of single stranded nucleic acid), a loop (a loop of single stranded nucleic acid), a T-shape formed of two hairpins, a bulge or similar. The capped end, however, can include more complicated structures within the cap such as multiple stem loops (forming a star shape), multiple hairpins, cross-arms, cruciforms, pseudoknots, G-quadruplexes or i-motifs. Such are used as the caps at the end of human chromosomes. This simple or more complex cap may be open or closed. If the cap is open, the terminal nucleotide(s) may be secured within the cap and/or the duplex, as discussed further below.

In order to be a targeting vector capable of delivery to a desired location, at least one of the caps includes a structural motif. Both caps may comprise a structural motif, each of which may be independently designed. The structural motif is a sequence which permits a desirable structure to be formed in the cap. Such a structure may be designed such that the structure forms under the relevant conditions, such as physiological conditions or the environment in which the vector is used (for example bacterial cell culture). The structural motif may act to stabilise the vector, such that it is more resistant to degradation for example. A stable vector is desirable to ensure that the gene or fragment thereof reaches the desired target intact, without changing. The structural motif may only form a structure under certain conditions, depending on the ionic strength and/or pH of their environment. If the vector is designed to only form a particular structure under a particular set of conditions, such as cellular conditions, it may be preferred that the capped end containing the structural motif is a closed end, which can therefore simply form a single stranded loop between the ends of the duplex under other conditions.

A closed capped end means that there are no terminal nucleotides that require securing. The closed end is continuous with both ends of the duplex. It may be preferred that at least one of the capped ends is closed, or that both capped ends are closed.

If the capped end is open, it is preferred that the terminal nucleotide(s) are secured to prevent immediate degradation.

The capped end may include a section of polynucleotide of any suitable length. The length of the capped end will depend upon the complexity of the capped end. If it is a simple hairpin, there is a minimal amount of sequence required to form the hairpin at the end of the duplex. However, for more complex structures such as multiple aptamers or G-quadruplexes, the capped end may be several hundred bases in length, such as up to 800 bases, or up to 700 bases, up to 600 bases or up to 500 bases in length.

Open Capped End

The vector of the present invention may have one or two capped ends that are “open”, such that the polynucleotide/vector is not continuous. Where open capped ends are present, each side of the nick/gap there is a free terminal nucleotide (5′ and 3′ terminal nucleotides). A nick is found between two adjacent nucleotides wherein the backbone is incomplete such that they are not linked. A gap occurs where there is one or more missing nucleotide(s) between the terminal residues, optionally wherein the terminal nucleotides are many nucleotides apart. This nick or gap occurs at or near the cap and not in the duplex section.

If the capped end is open, it is preferred that the terminal nucleotide residues are hydrogen-bonded intramolecularly to another part of the vector, including the structural motif if present at that end. Thus, for example the terminal nucleotide of the capped end (either 3′ or 5′) may be base paired to another residue in the capped end, and the terminal residue of the duplex (5′ or 3′) is base paired within the duplex. In one aspect, the terminal nucleotides form a base-pair with other nucleotides in the construct. Effectively, the vector ensures that there are no free single strands of nucleic acid with a terminal nucleotide available for an exonuclease to degrade.

One or more of the terminal residues may, however, be free from hydrogen bonding or more particularly base-pairing. In this instance, the capped end secures the terminal nucleotide by embracing, encircling or surrounding the terminal nucleotide, such that it is not free for a single strand nuclease to cleave it from the adjacent nucleotide in the construct (and then cleave the adjacent nucleotide and so on). In other words, the end is sterically protected from degradation, as it is not possible for larger entities to reach it. As an example, terminal nucleotides may be secured within a quadruplex motif.

In a further aspect, each terminal end may be secured by the formation of a duplex including at least the terminal residue. The duplex is formed by base-pairing between nucleotide sequences. These sequences may be adjacent (hairpin) or separated (stem loop etc.).

A residue refers to a single unit that makes up a nucleic acid polymer, such as a nucleotide. A terminal residue is a residue at the termini of the nucleotide strand at either the 3′ or 5′ end.

The end may be secured within conformations/structures such as quadruplexes.

Quadruplexes are quadruple (four stranded) structures, which may be involved in the structure of telomere ends of chromosomes. The underlying pattern is a tetrad, a planar arrangement of 4 residues, stabilised by Hoogsteen hydrogen bonding and coordination to a central cation. A quadruplex is formed by stacking of multiple tetrads. Many different topologies may form depending upon how the sequence initially folds into these arrangements. The quadruplex structure may be further stabilized by the presence of a cation, especially potassium. Quadruplexes have been shown to be possible in DNA, RNA, LNA, and PNA, and may be intramolecular.

Exemplary quadruplexes include G-quadruplexes, which are formed from G-rich sequences and i-motifs (intercalated motif) formed by cytosine-rich sequences.

In one aspect, therefore, the terminal nucleotide is secured within a quadruplex, optionally a G-quadruplex or an i-motif.

Structural Motif

The structural motif is designed such that it is formed from a single strand of nucleic acid. The structural motif has a sequence which permits it to form a structure, and this structure is preferably a secondary structure formed from a single strand of DNA. This structure may be described as a folded single strand of nucleic acid, since it is one strand of the duplex that extend out to form the capped end. In closed end configuration, this single strand then forms the opposite, complementary strand of the duplex. Thus, a closed end when in an unfolded configuration is a single strand of nucleic acid looped between the ends of a duplex. It may be possible that under certain conditions, such as storage conditions, the structural motif is present simply as a loop of single strand. The structure may then reform under conditions appropriate for use, such as physiological conditions.

The structure the motif forms may be achieved by the bases of the nucleic acid interacting with each other. The structure may include intramolecular hydrogen bonds in order to hold the motif into a structure. Suitable interactions and bonds holding the structure in place are described further herein.

The structural motif may form any suitable structure or conformation. Many structures are possible based upon a single strand of nucleic acid. Such include hairpins, stems, stem loops, loops, bulges, T-shapes (paired hairpins) and cruciforms. More complex structures may also be achieved, such as triplexes (three strands of nucleic acid, which could be intramolecular), a G-triplex, a quadruplex, an i-motif, a pseudoknot or any combination thereof.

It is possible to design a structural motif by including appropriate regions of complementary sequence within the single strand. Complementarity is defined herein.

Depending upon the sequence and other conditions, nucleic acids can form a variety of structural motifs which is thought to have biological significance.

Hairpins are formed when two regions of the same strand, usually complementary in nucleotide sequence when read in opposite directions, base-pair to form a duplex. A palindromic nucleotide sequence is capable of forming a hairpin. A hairpin may be entirely complementary, but due to steric hindrance, a few base pairs at the tip of the hairpin may be unpaired. The hairpin may include a few bases of non-complementary sequence at the tip.

Stem loop intramolecular base pairing is a pattern that can occur in single-stranded nucleic acid. The structure is also known as a hairpin loop. It occurs when two regions of the same strand, usually complementary in nucleotide sequence when read in opposite directions, base-pair to form a double helix that ends in an unpaired single stranded loop.

A pseudoknot is a nucleic acid secondary structure containing at least two stem-loop structures in which half of one stem is intercalated between the two halves of another stem.

Cruciform nucleic acid is a structure that requires at least a 6 nucleotide sequence of inverted repeats to form a structure consisting of a stem, branch point and loop in the shape of a cruciform.

G-quadruplex secondary structures (G4) are formed in nucleic acids by sequences that are rich in guanine. They are helical in shape and contain guanine tetrads that can form from one or more strands. I-motifs are four-stranded quadruplex structures formed by cytosine-rich DNA, similar to the G-quadruplex structures. C-rich DNA regions are common in gene regulation portions of the human genome.

i-motifs (intercalated-motif DNA), are cytosine-rich four-stranded quadruplex DNA structures, similar to the G-quadruplex structures.

Triplex DNA (also known as H-DNA or Triple-stranded DNA) is a DNA structure in which three oligonucleotides wind around each other and form a triple helix. In triple-stranded DNA, the third strand binds to a B-form DNA (via Watson-Crick base-pairing) double helix by forming Hoogsteen base pairs or reversed Hoogsteen hydrogen bonds.

The structural motif therefore permits the nucleotide to form a non-canonical structure. This structure is important in the context of the function of the structural motif, to provide the binding motif. The binding motif is described as having a “conformation” to prevent confusion between the various parts, but the terms conformation, structure, secondary structure, tertiary structure, configuration or geometry may all be used interchangeably. To confirm, a double helix or B-DNA is a DNA structure with canonical Watson-Crick base pairs.

The structural motif comprises a sequence that is capable of forming intramolecular hydrogen bonds. These hydrogen bonds may be base pairs of any kind, or Hoogsteen type hydrogen bonds seen in structures such as tetraplexes/quadruplexes.

Notably, a structural motif may be a sequence that includes one or more sections of sequence that are capable of forming base-pairs to another section of sequence.

The structural motif may therefore simply include two sections of sequence that are “complementary” and that base-pair to form an antiparallel or indeed parallel duplex. This duplex may or may not include the terminal residue (i.e. 3′ or 5′ end) of the strand. In this instance, the structural motif may form a hairpin (the two sections are contiguous) or stem loop (if the two sections are separated by a spacer sequence leaving single stranded nucleic acid). It will be understood that such a structure may be achieved by including an inverted repeat sequence in the structural motif. A palindromic sequence is a section of double stranded nucleic acid sequence wherein reading 5′ to 3′ forward on one section matches the sequence reading 5′ to 3′ forward on the complementary section with which it forms a duplex.

The structural motif may therefore include sequences necessary for the formation of one or more of: hairpins, stem loops, or pseudoknots. All of these conformations have in common two sections of sequence which can form a duplex. Alternative structures include lariats or lassos, which also include sections of sequence which can form a duplex.

The structural motif may be a triplex. In such, three oligonucleotides wind around each other and form a triple helix. In triple-stranded DNA, the third strand binds to a B-form DNA (via Watson-Crick base-pairing) double helix by forming Hoogsteen base pairs or reversed Hoogsteen hydrogen bonds. Triplex DNA is also called H-DNA. It can be formed intramolecularly when three sections have appropriate sequences. In some instances, the triplex may be formed using a duplex within the vector and an additional triplex forming oligonucleotide is added, and thus the triplex is intermolecular. The triplex can be a hybrid between DNA and RNA strands.

The structural motif can be a hybrid of different conformations or structures.

There are certain prerequisites for formation of structures based upon the sequence, length and orientation of the strands, along with the conditions. The hydration of the nucleic acid and the presence of various ions and/or ligands may also affect the structure of the nucleic acid. For example, at more acidic pH, i-motifs are more likely to form, whilst it may be single stranded at alkaline pH or neutral. Quadruplex motifs may form simpler hairpin structures at lower salt concentrations whilst they will adopt the G-quadruplex format when in the potassium ions at physiological pH.

Some of the sequence requirements for the formation of structure are detailed with some exemplary sequences in table 1 below:

TABLE 1

| Structure | Exemplary sequence 5′-3′ |
|---|---|
| Parallel stranded DNA | Purine rich (dG*dA)ₙ |
| (stabilised by Hoogsteen | CCTATTAAATCC |
| bonds or reverse Watson- | AAAAAAAAAATAATTTTAAATATTT |
| Crick bonding) | |
| Hairpin | (CAG)ₙ/(CTG)ₙ |
| | TGGGGCCCCA (hairpin and duplex) |
| Cross-arm | ATGGTCTTGCATGCAAGGCCATATATGGCACCAT |
| Triplex | (AAG)₅ (intermolecular triplex) |
| | C₂TC₅TC₂T₅G₂AG₅AG₂T₅G₂AG₅AG₂ |
| i-motif | CCCCTAACCCTAA (bimolecular) |
| | (CCCTAACCCCTAA)₂ (unimolecular) |
| Quadruplex | AG₆AG₃AG₃TG₂ (dimeric parallel strand) |
| | GGTTGGTGTGGTTGG (antiparallel unimolecular) |
| | TTAGGGTTAGGG (antiparallel tetramer) |

All of these structures have been documented as forming in physiological conditions.

The structural motif effectively provides the sequence permitting the formation of the capped end. Thus, it may be up to 800 nucleotides in length, up to 700, up to 600 or up to 500 nucleotides in length. A minimal structural motif may include about 12 nucleotides, such that a hairpin of 6 base pairs may be formed, together with a minimal section of binding motif, preferably at least 5 nucleotides in length.

When designing a suitable sequence for the structural motif, those skilled in the art will appreciate that some care needs to be employed to avoid using significant sequences in the structural motif and the duplex that are complementary, since this would interrupt the formation of the duplex and capped ends in the correct orientation, particularly when the vector is in preparation from a single stranded starting molecule. Those skilled in the art will be aware that the structure of a sequence may be checked with appropriate software, including at https//rna.urmc.rochester.edu/RNAstructureWeb/Servers/Predict1/Predict1.html

The structural motif includes at least one binding motif. The different structures described herein may be competent to form a binding motif. For example, a quadruplex is a structural motif which includes G rich loops that are the binding motif for nucleolin.

Hydrogen Bonding and Base Pairing

Hydrogen bonding is a non-covalent type of bonding between molecules or within them, intermolecularly or intramolecularly. These bonds are formed from an electronegative atom (the hydrogen acceptor) and a hydrogen atom that attaches covalently with another electronegative atom (the hydrogen donor may be nitrogen, oxygen, or fluorine atoms, although weaker hydrogen bonds may be formed with other donors) of the same molecule or of a different molecule. They are the strongest kind of dipole-dipole interaction. Hydrogen bonds are responsible for specific base-pair formation in a DNA double helix and are a factor to the stability of a DNA double helix structure.

Typically, in Watson-Crick base-pairing, hydrogen bonds form between the nitrogenous bases of the nucleotides (nucleobases). In standard base pairings, which are adenine-thymine (A-T) in DNA, adenine-uracil (A-U) in RNA and cytosine-guanine (C-G) in both, hydrogen bonds form. The A-T/U and C-G pairings function to form double or triple hydrogen bonds between the amine and carbonyl groups on the complementary bases.

A wobble base pair is a pairing between two nucleotides in nucleic molecules, most notably in RNA, that does not follow standard Watson-Crick base pair rules. The four main wobble base pairs are guanine-uracil (G-U), hypoxanthine-uracil (I-U), hypoxanthine-adenine (I-A), and hypoxanthine-cytosine (I-C). The thermodynamic stability of a wobble base pair is comparable to that of a Watson-Crick base pair. Wobble base pairs are fundamental in RNA structure.

Alternative or non-canonical base-pairings are also possible in nucleic acid structures, again held together by hydrogen bonds. These are generally more common in RNA, but are also possible in DNA and other nucleic acids. One example of non-canonical base pairing is Hoogsteen and reverse Hoogsteen base-pairing. In these interactions, the purine bases, adenine and guanine, flip their normal orientation and form a new set of hydrogen bonds with their partners. Hoogsteen hydrogen bonding has been shown to be present in quadruplexes such as the i-motif and G-quadruplex discussed in more detail herein.

A combination of various base-pairing mechanisms can also be envisaged. For example, when the hydrogen bonds in the A-T and G-C base pairs in canonical B-form DNA are formed, several hydrogen bond donor and acceptor groups in nucleobases remain unused. Each purine base has two such groups on the edges that are exposed in the major groove. Triplex DNA may form intermolecularly, between a duplex and a third oligonucleotide strand. The third strand bases may form Hoogsteen-type hydrogen bonds with purines in the B-form duplex.

Base-pairs may also form between natural and non-natural bases, and also between pairs of non-natural bases.

The intramolecular hydrogen bonds may also be interactions which are not defined as classical base pairing, such as the planar arrangement of guanine residues in the G-tetrad of a G-quadruplex, which is stabilised by Hoogsteen hydrogen bonding. These structures are discussed further below.

Further, stabilisation of nucleic acid molecules may also rely upon base-stacking interactions. Pi-pi stacking (also called π-π stacking) refers to attractive, noncovalent interactions between aromatic rings, since they contain pi bonds. These interactions are important in nucleobase stacking within nucleic acid molecules, which have been brought together by hydrogen bonding. It is thus likely that the single stranded nucleic acid constructs are further stabilised by base-stacking interactions. Other interactions stabilising the nucleic acid are also possible, these include pi-cation interactions, Van der Waals interactions and hydrophobic interactions.

All of these interactions and bonds may exist in any type of capped end of the duplexed section according to the present invention, in the simple or complex capped end or in the structural motif is present.

Two nucleotide sequences can be considered to be substantially complementary when the two sequences hybridise to each other under stringent conditions. In some embodiments, two nucleotide sequences are considered to be substantially complementary when they hybridise to each other under highly stringent conditions.

Stringent hybridisation conditions in the context of nucleic acid hybridisation are sequence dependent, and are different under different conditions. The hybridisation of nucleic acids is described in detail in Tijssen Laboratory Techniques in Biochemistry and Molecular Biology-Hybridization with Nucleic Acid Probes part I chapter 2, Elsevier, New York (1993) herein incorporated by reference. The stringency is determined by the hybridisation temperature and the salt concentration (high temperature and low salt is more stringent). For sequences that are not entirely complementary, the stringency must be reduced to a level that allows imperfect hybrids to form. If the stringency of the hybridisation is too low, then too much non-specific binding will occur and the desired vector will not be formed or maintained, and such low stringency conditions are not desirable in the context of the present invention.

In general, highly stringent hybridisation conditions are selected to be about 5° C. lower than the melting point (Tm) for the specific sequence at a defined ionic strength and pH.

The Tm is the temperature (under defined ionic strength and pH) at which 50% of the target sequence hybridises to a perfectly matched sequence (complementary). Very stringent conditions are selected to be equal to the Tm for a particular set of complementary sequences.

Suitable conditions for hybridisation can be used, for example the conditions for PCR primer annealing would be appropriate. The hybridisation may occur at temperatures of 45 to 65° C., optionally 50 to 55° C.

Quadruplexes

The sequences of G-quadruplexes are varied and may be defined by the putative formula: (G₃₊N₁₋ₙG₃₊N₁₋ₙG₃₊N₁₋ₙG₃₊) where N is any nucleotide, including guanine. The number of residues between the Guanines defines the lengths of the loops. Loops larger than 7 nucleotides have been seen. G-quadruplexes are highly polymorphic in nature. Both right and left hand quadruplexes have been reported in nature.

Quadruplexes (alternatively called tetraplexes) may complex around a central ion, for example. A number of ligands, both small molecules and proteins, can bind to quadruplexes. These ligands can be naturally occurring or synthetic. It has been found that all characterized G-quadruplex binding proteins share a 20 amino acid long motif/domain (RGRGR GRGGG SGGSG GRGRG—SEQ ID No. 1) called NIQI (Novel Interesting Quadruplex Interaction Motif) which is similar to the previously described RG-rich domain (RRGDG RRRGG GGRGQ GGRGR GGGFKG—SEQ ID No. 2) of the FM R1 G-quadruplex binding protein. Cationic porphyrins have been shown to bind intercalatively with G-quadruplexes. It may be important to match the quadruplex which has stacked quartets and the loops of nucleic acids holding it together. π-π interactions may be important determiners for ligand binding. Ligands should have a higher affinity for parallel folded quadruplexes. Ligands that bind to other structural motifs to stabilise them are also contemplated.

i-Motif

At least two parallel cytosine-rich strands which form a duplex are intercalated in antiparallel orientation which results in the formation of an “intercalated-motif”. Such structures may be formed by 1, 2, 3 or 4 strands, and each will differ in terms of strand orientation, sequence lengths and number of C:C⁺ base pairs. Generally, such structures are stabilised in acidic conditions. Various ligands have been designed to stabilise i-motifs such that they may operate in physiological conditions.

Cross Arm

A cross-arm structure is formed by nucleic acids with inverted repeats and involves intrastrand base pairing. In DNA, it is generally embedded in an AT rich area. These arms may form at an acute angle. A T-shaped hairpin is an example of a cross-arm structure.

Hairpin

Sequences with inverted repeats (IRs) or palindromes lead to the formation of hairpins. Hairpins may have a small loop of unpaired bases at the end/tip, even if the sequences are entirely complementary. Hairpins may be composed of any suitable inverted repeat sequences.

Bubble or Bulge

Such structures are formed in duplex nucleic acids where one strand includes unpaired nucleotides that bulge out as a single strand. This can happen on one or both sides of the duplex. These occur naturally in transcription bubbles.

Triplex DNA

Triplexes form between an oligopurine-oligopyrimidine duplex and a third strand in a sequence specific manner via Hoogsteen or reverse Hoogsteen bonds. Triplexes can be purely DNA, purely RNA or a hybrid of the two. Formation of triplex structures depends on several factors such as oligonucleotide length, base compositions, pH, presence of divalent cations and temperature. Triplexes have been detected in human cells and therefore will form under physiological conditions.

Binding Motif

The structural motif includes one or more binding motifs. The binding motifs are responsible for the targeting of the vector. The binding motif permits the interaction of the vector with a desired target in order to assist in the delivery of the vector to the desired location. Since the binding motif is included within the structural motif, it forms part of that structure. The binding motif is thus capable of assuming a conformation within the structural motif. In other words, the binding motif has a shape, a form, a geometry or a configuration. Such a conformation is important for the functioning of the binding motif. The conformation alone may be sufficient to ensure binding of the motif to the target.

Whilst the conformation of the binding motif is dependent upon the sequence of the motif, it is not simply the sequence per se that is responsible for the activity of the binding motif. Thus, the effect is not due to the nucleic acid sequence hybridising to a autologous complementary nucleic acid sequence during delivery, or the recognition of a consensus DNA sequence which can be present in duplex DNA.

The specificity of the binding motif may be due to a combination of the presence of particular residues and conformation that is important—for example the G residues in the quadruplex loops for binding to nucleolin.

Thus, the activity of the binding motif may be due to conformation alone, or a combination of conformation and the position of one or more residues within the structure. These residues may be described as key or specific residues, such as the G residues in the quadruplex loops.

The binding motif may comprise any combination of any one or more of the structures/conformations described herein. For example, the DNA aptamer that binds to thrombin has the sequence d(GGTTGGTGTGGTTGG) and has been noted to form a folded structure in solution, composed of two guanine quartets connected by two T-T loops spanning the narrow grooves at one end and a T-G-T loop spanning a wide groove at the other end. G quartets are the square planar structures also called a G tetrad and these structures form in G quadruplexes. Thus, this particular aptamer requires quartets and loops.

The binding motif is present within the structural motif since the structure is important for the binding motif's function. For example, as can be seen from FIG. 10, the binding motif (in this instance 207) fits between two sequences which form a stem structure (205 and 206). Thus, the entire structural motif comprises the binding motif plus a stem in this example. In this example, the structural motif provides support to the binding motif, acting as a scaffold, and also provides stability at the capped end. The structural motif may substantially include the one or more binding motifs, such that a minimal number of residues are required to support the binding motif in the vector. For example, a quadruplex may provide a structural motif with the loops of said quadruplex providing said binding motifs.

If an array of binding motifs are present, each of these may be separated by intervening sequences to enable each binding motif to form the correct conformation. In order to ensure proper folding of the array these intervening sequences can form branching stems of unique sequences, or can be otherwise designed to enforce the independent folding of each of motif and to limit the folding of the array to a single conformation.

The binding motif may be an aptamer. Aptamers are oligonucleotides that bind to a specific target molecule. In general, an aptamer has a unique structure and potential target binding capability. These features make aptamers high affinity (in nM to pM range) and specific binding molecules, able to differentiate between targets that differ by only one functional group. Aptamers may be termed “nucleic acid antibodies”. They are capable of binding to defined targets ranging from small molecules and proteins to whole calls or bacteria. Aptamers have been defined that are capable of binding to cancer cells (Tawiah et al, Biomedicines 2017, 5, 51, 5030051, herein incorporated by reference)

Aptamers may usually be created by repeatedly selecting them from a large random sequence pool, but natural aptamers also exist in riboswitches, for example. Nucleic acid aptamers are nucleic acid species (antibody mimics) having selectivity comparable to antibodies for a given target generated via in-vitro selection or equivalently, SELEX (systematic evolution of ligands by exponential enrichment) against their target. This can range from a small molecule to a cell. Aptamers may bind to their cognate target through various non-covalent interactions such as electrostatic interactions, hydrophobic interactions, conformational selection and induced fitting. The variability in aptamer sequences is what provides their versatility. The way aptamers fold, the order of the nucleic acids and the conditions of the environment they are in, all contribute to binding a target. Aptamers may offer discriminate recognition, but have advantages over antibodies as they can be engineered completely in vitro, are readily produced by chemical synthesis, and elicit little or no immunogenicity in therapeutic applications.

Generally aptamers are usually provided as single stranded nucleic acids, which results in a rapid clearance from the human body, for example, but the present invention effectively stabilises the aptamer, protecting it from immediate degradation, by including it within a larger vector.

Aptamers may be designed to bind to any suitable target, either on a cell surface or within a cell. Examples include a cell surface receptor or a nuclear transport component. The target may be as defined herein.

The binding motif may be a triplex. Triplexes are as described herein.

The binding motif may be a quadruplex. Quadruplexes are discussed herein. Formation of a quadruplex requires stacked G-tetrads or C-tetrads which are formed by the planar assembly of four residue using eight Hoogsteen hydrogen bonds thus making these structures highly thermally stable. A stabilizing metal cation may be included, or alternatively a stabilizing small molecule may be employed. Such are described in Maleki et al, Nucleic Acid Research, 47(20), 10744-10753, 2019, and Gonçalves et al, Chem Commun, 2006, 7 (45), 4685-4687, herein both incorporated by reference.

Quadruplexes may each have unique distinguishing features. Their uniqueness can be seen in their distinct folding patterns. These inherent differences in the folding patterns may involve changes in the loop connectivity and stabilizing metal cations, which result in differences in the groove structures. The differences in the groove widths and shapes offer opportunities for designing binding capabilities. Quadruplexes are capable of binding to specific targets. Quadruplexes may be employed to target the vector to the nucleus of a cell.

The binding motif may be a catalyst, such as a ribozyme or deoxyribozyme (DNAzyme). Catalytic nucleic acids are programmable in structure, easy to modify, and more stable; especially those that are comprised of DNA. They may be designed to be specific for a target in much the same way as aptamers. Catalytic nucleic acids are known to those skilled in the art. Catalytic nucleic acids are programmable in structure, easy to modify and DNA enzymes tend to be more stable than their protein counterparts. Similar methods to the development of aptamers can be used to develop catalytic nucleic acids. The first DNAzyme reported is called GR5 and is designed for RNA cleavage whilst only having 15 nucleotides in the active site. DNAzymes may be included within a G-quadruplex or triplex structure. Such are described in Ma & Liu, iScience 23, 100815, 2020, incorporated here by reference.

The binding motif may be any appropriate mixture of structural or conformational elements. The binding motif may rely upon one or more key or specific residues in particular locations to provide the binding specificity.

The binding motif may be a section of single stranded nucleic acid, which is held in place by the structural motif. An example of such a structure is a loop in a G-quadruplex. A complementary sequence for the single strand is therefore absent from the vector. This prevents a competition between the formation of the binding motif and the formation of a duplex.

As mentioned previously, it is the conformation, or the combination of conformation and the presence of particular (specific or key) residues that imparts the ability of the binding motif to selectively bind to its target entity. Thus, the binding motif does not target the vector for delivery based solely upon complementarity to a nucleotide sequence within the desired location.

The conformation or non-linear information content of the binding motif is therefore important. Consensus sequences, defined further below, rely on linear information content and not conformation. This means that the binding motifs are more amenable to modifications in sequence so long as the structure is maintained.

Furthermore, for the avoidance of doubt, the binding motif is not a consensus sequence that is conformation independent. For example consensus sequences are present in double stranded DNA that allow binding of proteins and enzymes to the DNA, such as restriction enzymes, methyltransferases, recombinases, transcription factors and the like. These tend to be short DNA sequences, typically 4-50 nucleotides in length. Sequence-specific DNA-binding proteins generally interact with the major groove of double stranded B-DNA, because it exposes more functional groups that identify a base pair. Thus, the sequence is usually recognised whilst the sequence is present in double or even sometimes single stranded DNA, without any non-canonical structure.

It is preferred that the binding motif is specific, such that the binding motif binds specifically to the desired target and not to any other component. The binding conditions are preferably physiological or the conditions in which the cell is maintained. The binding may be specific enough to distinguish between a modified and unmodified target, for example post translational modifications such as glycosylation, ubiquitination, methylation and the like. Aptamers have been shown to be specific enough to bind only to an unmodified target, for example.

There may be a sole binding motif within the structural motif, but it is preferred that there are a plurality of binding motifs, such as 1, 2, 3, 4, 5, 6, 7, 8, 9 or 10 binding motifs, preferably 2 to 5, 2 to 4 binding motifs. These binding motifs may be the same or different. If they are different they can bind to different entities, or more preferably they each bind to a different part of the same target. In this way it ensures specific targeting is achieved.

An array of binding motifs may be provided. An array comprises three or more binding motifs, each of which may be the same or different. The binding motifs in the array may be suitable separated by linker or spacer sequences in order for the motifs to fold.

Binding affinity can be defined as the strength of the binding interaction between the vector (in this case the binding motif) and its ligand/binding partner. Binding affinity is typically measured and reported by the equilibrium dissociation constant (K_{D}); this defines the likelihood that an interaction between two entities will break. The smaller the K_{D} value, the greater the binding affinity of the binding motif for its target. The larger the K_{D} value, the more weakly the binding motif and target are attracted to and bind to one another. Ligand binding assays may be performed, preferably under equilibrium conditions. Protocols for determining K_{D} are well known to those skilled in the art, and include diverse techniques such as gel-shift assays, pull-down assays, equilibrium dialysis, ELISA, analytical ultracentrifugation, bio-layer interferometry, surface plasmon resonance (SPR), and spectroscopic assays. The K_{D} is obtained from the ratio of K_{off} to Kₒₙ (i.e. K_{off}/Kₒₙ) and is expressed as a molar concentration (M).

Binding affinity is influenced by non-covalent intermolecular interactions such as hydrogen bonding, electrostatic interactions, hydrophobic and Van der Waals forces between the two molecules. Thus, the careful design of binding motifs makes it possible to bind to desired targets.

The specificity of binding under optimal/physiological conditions can be defined as uniformity of targets that can be bound. The higher specificity the more uniform targets that can be bound. Highly specific aptamers can distinguish not only the type of target but also a modification that has been made. By contrast, a promiscuous binding motif would bind to various related structures. In specific embodiments, a binding motif that specifically binds to a target is intended to refer to a motif that binds to a target with a K_{D} of 1 mM or less, 100 nM or less, 10 nM or less, or 3 nM or less. It may be possible to select a binding motif that binds with a K_{D} in the picomolar or even femtomolar range in future.

A competitive assay may be used to determine the ability of the motif to bind to its target.

As used herein, the term specific binding refers to an ability of the motif to discriminate between possible binding partners in the environment in which binding is to occur. A binding motif that interacts with one particular target when other potential targets are present is said to “bind specifically” to the target with which it interacts. In some embodiments, specific binding is assessed by detecting or determining degree of association between the binding motif and its target; in some embodiments, specific binding is assessed by detecting or determining the degree of dissociation of a binding motif-target complex; in some embodiments, specific binding is assessed by detecting or determining the ability of the binding agent to compete in interactions between its target and another entity. In some embodiments, specific binding is assessed by performing such detections or determinations across a range of concentrations.

Binding Motif Target

The binding motif is preferably specific for a target, or is capable of binding specifically to a target. This target may be any suitable or desirable target. Binding motif as used herein is meant to refer to a targetable nucleic acid which is capable of binding to a target. The binding motif is part of a structural motif. The vector includes at least one binding motif that is specific for a cellular target. The vector may optionally include a separate binding motif that is specific for a non-cellular target, to enable the vector to be used to carry a non-DNA payload, if required.

The target as used herein is meant to refer to any compound or entity that may be capable of binding to or otherwise interacting with one or more binding motifs. Examples of targets include peptides, proteins, modified proteins, glycoproteins, peptidoglycans, lipids, phospholipids, glycolipids, nucleic acids, and/or cholesterol. The target may also be called a target entity or simply an entity.

The cellular target may be present on the surface of a cell, for example a membrane protein, a receptor, a ligand, a sugar, a glycosylated protein, a peptidoglycan, a lipid, a phospholipid or glycolipids.

Targeting a cell surface target is desirable, since in multicellular organisms, it enables the direction of the vector to a particular tissue or cell type by selection of an appropriate marker/target. The cell type may be cancerous and the target may only be expressed on such cells. This may permit the specific delivery of a cytotoxic gene or fragment thereof only to cells which are desirable to remove.

Alternatively, the binding motif can permit the targeting of the vector to a particular tissue type, for example, the myocardium to treat heart diseases.

The cellular target may be at the blood brain barrier, assisting the vector across the blood brain barrier. Aptamers have already successfully been shown to target and cross the blood brain barrier.

The cellular target may be present within a cell. For a eukaryotic cell, the target may be present on an internal membrane of the cell, surrounding an organelle, such as the nuclear membrane, mitochondrial membrane, endoplasmic reticulum (ER) membrane, the Golgi apparatus membrane, and the lysosomal membrane. Each of the internal membranes is unique and therefore these differences can be exploited to enable targeting to these particular organelles.

Targeting the nucleus is desirable for eukaryotic cells since it permits the delivery of the vector to an appropriate location for expression. The nuclear membrane is also termed the nuclear envelope as it is a double membrane. The target may be part of the nuclear pore complexes (NPCs). The target may be a cellular nuclear transport protein such as importin-α and importin-β. The target may be any part of the nuclear transport system. Nuclear targeting may be achieved by the inclusion of a quadruplex. Targeting the nucleus may also be achieved by designing the binding motif to target histones and the like. Targeting the nuclear matrix may also be advantageous. Many proteins may be associated with the nuclear matrix, such as the Scaffold, or Matrix Associated Proteins (SAR or MAR), which are thought to have a role in the organisation of chromatins. Such targeting may be advantageous if it is desired to localise the vector into transcriptionally active spots within the nucleus.

The cellular target may be part of the endosomal system, thus assisting transport in or out of cells.

Alternatively, cytoplasmic components such as proteins and inclusions may be targeted. For example, this may permit the specific targeting of cells with undesirable cytoplasmic components, such as prions or proteinaceous plaques.

The cellular target may be present on the mitochondria, to permit expression within the mitochondria. This may be relevant for mitochondrial-linked diseases.

Nuclear targeting is advantageous in that it may permit a reduction in the amount of vector required for a therapeutic dose, since more of the gene or fragment thereof is delivered to the location where expression is desired. Further, for applications such as vaccines, it may permit an earlier expression of the gene or fragment thereof, providing a more rapid response from the immune system.

Nucleic acid generally enters the nucleus through the nuclear pore complex (NPC); this is an aqueous channel in the nuclear envelope which is large and formed of numerous proteins. Entry through NPCs may be size dependent, with smaller vectors being able to localise to the nucleus more quickly. Other technology may attach an NLS peptide to attempt to target vectors through the NPC, although such an approach has had varied levels of success. Proteins such as transcription factors may be present in the cytoplasm ready to be trafficked to the nucleus, depending on the type of cell and/or developmental stage of the cell. Furthermore, the abundance and expression of transcription factors may be variable. It is preferred to target entities commonly trafficked to the nucleus to ensure nuclear delivery. Such targets include histones, nucleolin, telomere binding proteins and the like. Such are preferred targets due to their more constitutive expression across different cell types and the demonstrated ability of these to permit nuclear targeting.

Cell-specific nuclear targeting may be possible, by targeting cell-specific proteins that are being trafficked to the nucleus.

Productive transfer of the vector may not only require cell entry, but also a number of cellular events that allow the vector to move from the cell surface, through the cytoplasm and ultimately across the nuclear envelope and into the nucleus. Intracellular trafficking components may therefore also provide a target for a binding motif. Several proteins and other molecules are involved with intracellular trafficking/cytoplasmic transport/nuclear import, these include, polyamines, nucleic acid binding proteins, microtubules, dynein, cationic proteins, chaperones and nuclear import proteins, telomere binding proteins, histones or nucleolin.

The cytoplasm is crowded with proteins, and it has previously been shown that nucleic acids which are larger than 2000 base pairs are unable to effectively diffuse through the cytoplasm in a useful time frame. Including a binding motif that binds to a component in the intracellular trafficking can therefore increase the speed of transfer across the cytoplasm, particularly for larger vectors.

When vectors enter cells they may be endocytosed, resulting in the vector being concentrated in endosomes. Ultimately, endosomes may be delivered to lysosomes, and the contents of the endosome are degraded. Therefore, it is not desirable to target the endosomal pathway in order to effectively target the vector. Therefore, the vector may either target cellular entry through a mechanism other than endocytosis (such as targeting a specific transporter) or include a mechanism for escaping the endosome. High efficiency of endosome escape may be achieved by choosing a receptor that exhibits one or more of the following properties: a highly expressed cell surface receptor (>10⁵), a fast receptor uptake (approximately 20 minutes is considered to be fast), and/or the receptor has an enhanced endosomal escape efficiency (approximately 1% cargo escapes). For example, the liver cell surface receptor ASGPR is expressed at high levels on the surface of liver cells (10⁶ receptors per cell) and internalises rapidly.

It is possible to select binding motifs that not only bind to targets but also internalise into cells. In relation to aptamers, for example, cell-based SELEX has been used to ensure that the aptamer is internalised after binding to the target.

Possible transmembrane receptors for targeting are included below; this list is not comprehensive:

TABLE 2

| | | | |
|---|---|---|---|
| Gene (Symbol | | | Endocytosis |
| (Full name)) | Ligand | Function | type |

G-protein coupled receptor (GPCR)

| | | | |
|---|---|---|---|
| 1. ADRB1 | Epinephrine, | Mediate | CDE (clathrin- |
| (Adrenoceptor β 1) | norepinephrine | catecholamines | dependent |
| | | action | endocytosis) |
| 2. ADRB2 | Epinephrine, | Mediate | CDE |
| (Adrenoceptor β 2) | norepinephrine | catecholamines | |
| | | action | |
| 3. ADRB3 | Norepinephrine | Mediate | CDE |
| (Adrenoceptor β 3) | | catecholamines | |
| | | action | |
| 4. CCR5 (Chemokine | CCl3, CCl4, | Leukocyte | CDE |
| (C-C motif) receptor 5 ) | CCl5, CCl8, | trafficking, | |
| | CCl13, CCl16 | angiogenesis, | |
| | | apoptosis | |
| 5. CXCR1 | CXCl6, CXCl8 | Leukocyte | CDE |
| (Chemokine (C-X-C | | trafficking, | |
| motif) receptor 1) | | angiogenesis, | |
| | | apoptosis | |
| 6. CXCR2 Chemokine | CXCl1, CXCl2, | Leukocyte | CDE |
| (C-X-C motif) receptor | CXCl3, CXCl5 | trafficking, | |
| 2) | CXCl6, CXCl7, | angiogenesis, | |
| | CXCl | apoptosis | |
| 7. CXCR4 | CXCl14 | Leukocyte | CDE |
| (Chemokine (C-X-C | | trafficking, | |
| motif) | | angiogenesis, | |
| 8. F2R Coagulation | Thrombin | platelets | CDE |
| factor II receptor) | | activation, | |
| | | vascular | |
| | | development | |

Receptor tyrosine kinase (RTK)

| | | | |
|---|---|---|---|
| 9. CSF1R (Colony | M-CSF, IL34 | Macrophage | CDE |
| stimulating factor 1 | | regulator | |
| receptor) | | | |
| 10. EGFR (Epidermal | EGF | Proliferation, | CDE/CIE |
| growth factor receptor) | | differentiation | ((clathrin- |
| | | | independent |
| | | | endocytosis) |
| 11. ERBB2 (Erb-b2 | EGF | Proliferation, | CDE |
| receptor tyrosine kinase | | differentiation | |
| 2) | | | |
| 12. ERBB3 (Erb-b2 | EGF | Proliferation, | CDE |
| receptor tyrosine kinase | | differentiation | |
| 3) | | | |
| 13. ERBB4 (Erb-b2 | EGF | Proliferation, | CDE |
| receptor tyrosine kinase | | differentiation | |
| 4) | | | |
| 14. FGFR1 (Fibroblast | FGF1, FGF2, | Proliferation, | CDE/CIE |
| growth factor receptor | FGF3, FGF6, | differentiation | |
| 1) | FGF7 | | |
| 15. FGFR2 (Fibroblast | FGF1, FGF4, | Proliferation, | CDE/CIE |
| growth factor receptor | FGF6, FGF7, | differentiation | |
| 2) | FGF8 | | |
| 16. FGFR3 (Fibroblast | FGF3, FGF4, | Proliferation, | CDE/CIE |
| growth factor receptor | FGF5, FGF6, | differentiation | |
| 3) | FGF7 | | |
| 17. FGFR4 (Fibroblast | FGF1, FGF3, | Proliferation, | CDE/CIE |
| growth factor receptor | FGF4, FGF5, | differentiation | |
| 4) | FGF9 | | |
| 18. FLT1 (Fms-related | VEGFA, | Angiogenesis | CDE/CIE |
| tyrosine kinase | VEGFB, PGF | | |
| 1)/VEGFR1 | | | |
| (Vascular endothelial | | | |
| growth factor | | | |
| receptor1) | | | |
| 19. IGF1R (Insulin-like | IGF1, IGF2 | Proliferation, | CDE |
| growth factor 1 | | differentiation | |
| receptor) | | | |
| 20. IGF2R (Insulin-like | IGF2, | Proliferation, | ICDE |
| growth factor 2 | Transferrin | differentiation | |
| receptor) | | | |
| 21. KDR (Kinase | VEGFA, | Proliferation, | CDE/CIE |
| insert domain | VEGFC | angiogenesis | |
| receptor)/VEGFR2 | | | |
| (Vascular endothelial | | | |
| growth factor | | | |
| receptor2) | | | |
| 22. MET (Tyrosine- | HGF | Proliferation, | CDE |
| protein kinase (met) | | angiogenesis | |
| 23. NTRK1 | NGF | Differentiation | CDE |
| (Neurotrophic tyrosine | | | |
| kinase receptor type 1) | | | |
| 24. PDGFRA (Platelet- | PDGFC | Proliferation, | CDE |
| derived growth factor a | | differentiation, | |
| receptor) | | | |
| 25. TGFBR1 | TGF-β | Proliferation | CDE/CIE |
| (Transforming growth | | tumor | |
| factor β receptor I) | | transformation | |
| 26. TGFBR2 | TGF-β | Proliferation, | CDE/CIE |
| (Transforming growth | | tumor | |
| factor β receptor I) | | transformation | |

Transmembrane receptor (TMR)

| | | | |
|---|---|---|---|
| 27. FOLR1 (Folate | Folic acid | Transport folic | CDE |
| receptor 1) | | acid | |
| 28. FOLR2 (Folate | Folic acid | Transport folic | CDE |
| receptor 2) | | acid | |
| 29. FOLR3 (Folate | Folic acid | Transport folic | CDE |
| receptor 3 | | acid | |
| 30. IL2RA (Interleukin | IL2 | Regulate | Indt |
| 2 receptor α) | | immune system | (Clathrin/ |
| | | | caveolin- |
| | | | independent |
| | | | endocytosis) |
| 31. IL2RB (Interleukin | IL2, IL15 | Regulate | Indt |
| 2 receptor β) | | immune system | |
| 32. IL2RG (Interleukin | IL2, IL-4, IL15 | Regulate | Indt |
| 2 receptor γ) | | immune system | |
| 33. LDLR (Low density | LDL, | Transport lipid | CDE |
| lipoprotein receptor) | ApoB100, | | |
| | ApoE, IDL | | |
| 34. TFRC (Transferrin | Transferrin, | Transport iron | CDE |
| receptor) | HFE | | |

Some suitable cell targeting aptamers are included below:

TABLE 3
exemplary aptamers that are capable of targeting.

| Aptamer | Target | Sequence | Internalised? | Structure |
|---|---|---|---|---|
| arahh001 | Present on | ACGTACCGACTTCGTATGCCAACAGCCCTTT | Yes | — |
| | primary tumour | ATCCACCTC | | |
| | endothelial cells | | | |
| TEPP | TfR and EpCAM | GCGCGGTACCGCGCTAACGGAGGTTGCGTC | Yes also | Two |
| | | CGT | crosses | stem |
| | | | blood brain | loops |
| | | | barrier | |
| MUC1 | Mucin (OVCAR-3, | GCAGTTGATCCTTTGGATACCCTGG | Unknown | — |
| | A549, pancreatic, | | | |
| | prostate, MCF-7, | | | |
| | HepG2) | | | |
| S1.3/ | Mucin (MCF-7) | GGGAGACAAGAATAAACGCTCAAGCAGTT | Unknown | — |
| S2.2 | | GATCCTTTGGATACCCTGGTTCGACAGGAG | | |
| | | GCTCACAACAGGC | | |
| 5TR-1 | Mucin (MCF-7) | GGGAGACAAGAATAAACGCTCAAGAAGTG | Unknown | — |
| | | AAAATGACAGAACACAACATTCGACAGGAG | | |
| | | GCTCACAACAGGC | | |
| SGC8 | Protein Tyrosine | ATCTAACTGCTGCGCCGCCGGGAAAATACT | Unknown | Stem |
| | kinase 7 (PTK7) | GTACGGTTAGA-(CH2)6-NH2 | | loop |
| | (CCRF-CEM-T | | | |
| | cell leukaemia) | | | |
| SGA16 | Protein Tyrosine | TTTAAAATACCAGCTTATTCAATTAGTCACA | Unknown | Two |
| | kinase 7 (PTK7) | CTTAGAGTTCTAGCTGCTGCGCCGCCGGGA | | stem |
| | (CCRF-CEM-T | AAATACTGTACGGATAGATAGTAAGTGCAA | | loops |
| | cell leukaemia) | TCT | | |
| SYL3C | EpCAM (MDA- | CACTACAGAGGTTGCGTCTGTCCCACGTTGT | Unknown | Two |
| | MB-231, Kato III, | CATGGGGGGTTGGCCTG | | small |
| | HT-29, T47D, cell | | | stem |
| | sorting aptamer | | | loops |
| | set) | | | |
| SYL1 | EpCAM; MDA-MB- | AGCGTCGAATACCACTACAGTTTGGCTCTG | Unknown | Dual G- |
| SYL2 | 231, Kato III, | GGGGATGTGGAGGGGGGTATGGGTGGGA | | quadruplex |
| SYL3 | HT-29, T47D | GTCAATGGAGCTCGTGGTCAG | | Stem |
| SYL4 | Reported as for | AGCGTCGAATACCACTACAGAGCTCGGGGT | | loop |
| | cell sorting | TTTTTGGGGTTTTTTGGGGTTTTGGTGGGGC | | Stem lop |
| | aptamer set | TAATGGAGCTCGTGGTCAG | | G- |
| | | AGCGTCGAATACCACTACAGAGGTTGCGTC | | quadruplex |
| | | TGTCCCACGTTGTCATGGGGGGTTGGCCTG | | on a |
| | | CTAATGGAGCTCGTGGTCAG | | stem |
| | | AGCGTCGAATACCACTACAGAGCTCCGGGG | | |
| | | TTTTTGGGGGTTTTTCTGGGGTTTTTTGGGG | | |
| | | CTAATGGAGCTCGTGGTCAG | | |
| TDO5 | IgG receptors; | AACACCGGGAGGATAGTTCGGTGGCTGTTC | Unknown | Stem |
| | Ramos cells | AGGGTCTCCTCCCGGTG-(CH2)6-NH2 | | loop |
| | (B-cell lymphoma) | | | |
| A1 | No data; A549 | GGTTGCATGCCGTGGGGAGGGGGGTGGGT | Unknown | G- |
| | cells | TTTATAGCGTACTCAG(CH2)6-NH2 | | quadruplex |
| AS1411; | Nucleolin; C6, | TTGGTGGTGGTGGTTGTGGTGGTGGTGG | Unknown | 2x G- |
| our | HeLa, Hep-G2, | | | quadruplex |
| candidate | Caco-2, U87MG, | | | |
| | F11, C6, CT-26 | | | |
| GMT4,8 | No data; CCRF- | TGACGAGCCCAAGTTACCTTGGTGATGGTT | | G- |
| | CEM, U87 | TTTGGTGGTAACGGGGGCGGGTGAGTAGA | | quadruplex |
| | | ATCTCCGCTGCCTACA | | |
| CSC1 | No data; DU145 | ACCTTGGCTGTCGTGTTGTAGGTGGTTTGCT | Unknown | — |
| | | GCGGTGGGCTCAAGAAGAAAGCGCAAAGG | | |
| | | TCAGTGGTCAGAGCGT | | |
| CSC13 | No data; prostate | ACCTTGGCTGTCGTGTTGTGGGGTGTCGTA | Unknown | — |
| | cancer stem cells | TCTTTCGTGTCTTATTATTTTCTAGGGGAGG | | |
| | | TCAGTGGTCAGAGCGT | | |
| KDED2a-3 | No data; DLD-1 | TGCCCGCGAAAACTGCTATTACGTGTGAGA | Unknown | Middle |
| | | GGAAAGATCACGCGGGTTCGTGGACACGG | | stem |
| | | TTTTTTTTTTT | | |
| KCHA10 | No data; HCT116 | ATCCAGAGTGACGCAGCAGGGGAGGCGAG | | — |
| | | AGCGCACAATAACGATGGTTGGGACCCAAC | | |
| | | TGTTTGGACACGGTGGCTTAGTTTTTTTTTTT | | |
| R13 | No data; A549 | TCTCTAGTTATTGAGTTTTCTTTTATGGGTG | | G |
| | | GGTGGGGGGTTTTT | | quadruplex |
| S6 | No data; SK-BR-3 | TGGATGGGGAGATCCGTTGAGTAAGCGGG | | 2x stem |
| | | CGTGTCTCTCTGCCGCCTTGCTATGGGG | | loops |
| GBI-10 | | GGCTGTTGTGAGCCTCCTCCCAGAGGGAAG | | — |
| | | ACTTTAGGTTCGGTTCACGTCCCGCTTATTC | | |
| | | TTACTCCC | | |
| A-1 | no data; HepG2 | TAACTCAATAAGCTAGGTGGGTGGGGGAC | | G- |
| | | ACTACCCGGGGGGTGGTTGGGT | | quadruplex |
| | No data; H23 cell | Short oligos | | — |
| | sorting | | | |

The following table depicts the targets selected by the present inventors and exemplified (see Examples). Histone H4 is one of the five main histone proteins involved in the structure of chromatin in eukaryotic cells.

TABLE 4
target candidates and binding motif structure.

| Target | Binding motif structure |
|---|---|
| H4-K16Ac | Stem loops |
| H4 | G-quadruplex |
| H4 (made for microscopy) | 2 large loops |
| H4 | Stem-loops |

The vector of the present invention may have a plurality of binding motifs and therefore may bind a plurality of targets. For example, it is possible to include not only a binding motif for a particular cellular target, but also one to a nuclear target ensuring not only specific cell-entry but also nuclear entry in that specific cell. It may be preferred in that instance that the binding motifs are on different ends of the vector. Alternatively, these different binding motifs may be present as an array at one end of the vector.

Delivery

The nucleic acid vector of the present invention may be described as a delivery vector, by virtue of the binding motifs directing the vector to a desired cellular target. Thus, the vector of the present invention may be a delivery vector, wherein the cellular targeting mechanism is included within the nucleic acid of the vector itself, by the inclusion of structural motifs comprising binding motifs.

The vector of the present invention may be used to deliver itself to a specified or desired cellular target. This cellular target may be in vivo or in vitro.

The vector may be prepared for such delivery without the use of chemical delivery agents, such as peptide sequences, liposomes and the like. This is because the vector provides and all-in-one solution, providing a minimal solution to an issue that has not completely been resolved. In other words, the vector may be provided as “naked” DNA. Naked DNA is an attractive non-viral gene vector because of its inherent simplicity and the low immunogenicity of DNA per se.

The vector of the present invention can be incorporated into pharmaceutical compositions suitable for administration to a subject for in vivo delivery to cells, tissues, or organs of the subject. Typically, the pharmaceutical composition comprises the vector of the present invention and a pharmaceutically acceptable carrier. For example, vectors of the invention can be incorporated into a pharmaceutical composition suitable for a desired route of therapeutic administration. Passive tissue transduction via high-pressure intravenous or intra-arterial infusion are potential therapeutic routes. Pharmaceutical compositions for therapeutic purposes can be formulated as a solution, micro emulsion, dispersion and the like. Sterile injectable solutions can be prepared by incorporating the vector in the required amount in an appropriate buffer with one or a combination of ingredients, as required, followed by filtered sterilisation.

The vector as disclosed herein can be incorporated into a pharmaceutical composition suitable for topical, systemic, intra-amniotic, intrathecal, intracranial, intra-arterial, intravenous, intra-lymphatic, intraperitoneal, subcutaneous, tracheal, intra-tissue (e.g., intramuscular, intra-cardiac, intra-hepatic, intra-renal, intra-cerebral), intra-thecal, intra-vesical, conjunctival (e.g., extra-orbital, intra-orbital, retro-orbital, intra-retinal, sub-retinal, choroidal, sub-choroidal, intra-stromal, intra-cameral and intra-vitreal), intra-cochlear, and mucosal (e.g., oral, rectal, nasal) administration.

Pharmaceutically active compositions comprising a vector can be formulated to deliver a transgene to the specified cells of a recipient, resulting in the therapeutic expression of the transgene therein. The composition can also include a pharmaceutically acceptable carrier.

The compositions and vectors provided herein can be used to deliver a transgene for various purposes.

A vector described herein can be administered to an organism for transduction of cells in vivo.

Suitable methods of administering such nucleic acids are available and well known to those of skill in the art. Exemplary modes of administration of the vector disclosed herein includes oral, rectal, trans-mucosal, intra-nasal, inhalation (e.g., via an aerosol), buccal (e.g., sublingual), vaginal, intra-thecal, intra-ocular, subdermal, transdermal, intra-endothelial, in utero, parenteral (e.g., intravenous, subcutaneous, intra-dermal, intra-cranial, intra-muscular, intra-pleural, intra-cerebral, and intra-articular), topical (e.g., to both skin and mucosal surfaces, including airway surfaces, and transdermal administration), intra-lymphatic, and the like, as well as direct tissue or organ injection. Direct injection may be relevant when the desired cells are muscle cells, for examples for vaccines.

Additionally, more than one transgene/coding sequence or can be included in a single vector, or multiple vectors within a composition.

Alternatively, cells may be removed from a subject, a vector is introduced therein, and the cells are then replaced back into the subject. Methods of removing cells from subject for treatment ex vivo, followed by introduction back into the subject are known to those skilled in the art. Alternatively, allogenic cells (from a different donor) may be modified and introduced to a subject.

The delivery may be specific to a target cell which is not a cell of the subject—for example a bacterial, fungal or parasite cell. In this instance, the vector may include toxic transgenes to assist in the removal of unwanted cell types.

Conditions

Nucleic acid structures can be affected by changes in conditions. The sequences for the structural motif can be selected such that the conformation is adopted under the conditions under which the nucleic acid construct is to be used (such as pH, temperature, salt concentration, pressure, protein concentration, sugar concentration, osmotic pressure and the like).

The vector can be used in many various conditions, such as physiological conditions or conditions that favour production of protein in microorganisms, for example.

Physiological conditions are conditions of the external or internal milieu that may occur in nature for that organism or cell system, and may be the appropriate conditions for the structural motif to assume the relevant conformation.

Additional stabilising entities may be employed to assist the folding of the caps/structural motifs. For example, G-quadruplexes may be stabilised using ions and small molecule ligands as described earlier. Stabilisers of triplexes include molecules with an extended aromatic ring structure as described in del Mundo et al, BBA—Molecular Cell Research 1866 (2019) 118539, herein incorporated by reference.

It is preferred that the sequences defined herein as complementary are capable of forming a duplex under physiological conditions.

Nucleic Acid Vaccines

The vector of the present invention is of particular use as a nucleic acid, optionally a DNA vaccine.

The vector may be used for expression in a host cell, particularly for production of an antigen. DNA or RNA vaccines typically encode a modified form or part of an infectious organism. DNA or RNA vaccines are administered to a subject where they then express the selected protein of the infectious organism, initiating an immune response against that protein which is typically protective. DNA or RNA vaccines may also encode a tumour antigen in a cancer immunotherapy approach.

The vector of the present invention may therefore be a vaccine composition. The composition may further comprise any adjuvant sequence to boost the immunogenic effect. A vaccine composition may be targeted to a suitable tissue that permits the easy expression of such a vaccine. Exemplary is muscle cell. If the vaccine is a cancer vaccine, the vector may be targeted to the cell type affected by the cancer to permit a localised response, for example a prostate cancer vaccine may be targeted to prostate cells.

Gene Therapy

The vector can be used to express a functional gene or fragment thereof where a subject has a genetic disorder caused by a dysfunctional version of that gene. Examples of such diseases are well known in the art. It may be desirable to target the expression of the gene or fragment thereof in the tissue, organ or cell type that is affected by the disease, for example express insulin in the pancreas.

Cytotoxin Delivery

The vector can be used to express a gene or fragment thereof which is cytotoxic to the cell. Such is desirable in the treatment of cancer. By targeting the vector specifically to cells expressing a cancer-related marker, the cell may be killed. Further, the same approach can be taken to have an antibacterial or antifungal approach. These could be pathogenic organisms which have caused an infection of a subject, be that human or animal, or be present within the environment or in industrial processes. By targeting the vector to a particular microorganism, that micro-organism may be selectively removed. This has a clear benefit therapeutically, since for example only the pathogenic bacteria will be targeted, and also environmentally and industrially, since contamination can also be cleared, for example cyanobacteria in bodies of water.

The cytotoxin may be any suitable protein or peptide that may induce cell death, either by apoptosis or cell necrosis. The cytotoxin may be one produced by the immune cells of the subject, or alternatively may be derived from a different species, such as a venom or toxin from a plant.

Therapeutic Uses

It is preferred for therapeutic human or animal uses in particular that the vector lacks a bacterial origin of replication, lacks resistance genes (i.e. for antibiotics), lacks prokaryotic patterns of methylation (except for vaccines where the same may be helpful), and is devoid of sequences that would identify the nucleic acid as foreign to the host cell.

Any possible therapeutic use of the vector is envisioned.

Additional Functions

The vector of the present invention may include modified nucleotides. As can be seen in a following section, as one method of making the vector includes using a polymerase enzyme, it is possible to feed the reaction with modified nucleotides. Modified nucleotides may form the attachment point for many other entities, such as small molecules, peptides, adjuvants, agonist, antagonist, immune-stimulants, markers, beacons, antibodies or fragments thereof and/or proteins. These entities may have a function within the cell and act to supplement the gene or fragment thereof provided by the vector, and/or to provide additional targeting. For example, a chemotherapeutic drug such as Paclitaxel may be attached to a cytotoxic gene for targeting a cancer cell.

Thus, the vector of the invention is capable of targeting a combined therapy to a desired location.

Should the vector be delivering a vaccine, it is possible to include either a sequence encoding an adjuvant, immune-stimulant or an agonist to the vector, or supply the same attached to the vector using attachment points described herein. This may be particularly relevant for cancer vaccines.

Alternatively, the vector may include additional binding motifs that are specific for entities such as small molecules, peptides, adjuvants, agonist, antagonist, immune-stimulants, markers, beacons, antibodies or fragments thereof and/or proteins. This would additionally permit the vector to ferry these entities to a desired location. In one embodiment, one of the capped ends can include binding motif(s) specific for a target, whilst the other capped end includes binding motifs which carry an entity such as a small molecule drug.

Further alternatively, this technology permits the association of markers and tracers to the vector, such as florescent nucleotides which allows for tracking.

The capped ends of the vector may also include additional sequences to provide further functions, such as the location for a primer binding site or a recognition sequence for a primase.

Modified nucleotides that are introduced to the vector may permit the linking of other entities or may contribute to charge modification or provide a traceable marker. Examples of such are described in table 5 below:

TABLE 5

| Novel Feature | Example of Nucleotide | Use/benefit |
|---|---|---|
| Click chemistry | Alkyne-modified | Attacking entities such as |
| compatible | nucleotides will react | antibodies, agonists, adjuvants |
| nucleic acid | with azide moieties to | |
| | form a triazole link | |
| Charge | Alpha-thiol nucleotides | Helping the passage through |
| reduction | | lipid membranes and/or escape |
| | | endosomes |
| Fluorescence | Cy5 or Cy3 coupled | Tracking and localising the |
| | nucleotides | vector in a cell |

The modified nucleotides can be included in any part of the vector. As can be seen from the unique method of manufacturing vectors, the modified nucleotides can be added to the duplexed section and/or indeed either or both of the capped ends.

Manufacturing the Vector

The vector described herein may be made using a unique method described here. The manufacturing method below is suitable for preparing a vector with two capped ends, at least one of which is a closed end. The method is highly efficient and enables large-scale production, by initially producing a single stranded intermediate which may be filled-in and closed. The method may be modified to produce an entirely covalently closed vector by the use of an enzyme to link the two free ends, such as a ligase. Alternatively, should two open caps be required, a specific nickase can be used to introduce a nick into the cap, and specific recognition sites for this nickase can be included in the vector.

The method of manufacturing the vector relies upon the amplification of a template nucleic acid by rolling circle amplification with a relevant polymerase enzyme, resulting in the production of a single stranded nucleic acid which includes numerous repeats of the template, otherwise called a concatemer. This single stranded nucleic acid concatemer may then then processed into the vector. Thus, the vector may be made via synthesizing a single strand of nucleic acid initially.

The method of the invention relies upon the formation of a base paired section in the single strand which permit cleavage with an appropriate enzyme, since the double stranded section of the hairpin permits enzyme binding and cleavage. This then separates each individual vector from the concatemer of many vectors.

The vector made by this method is a vector with a section of duplex (a single strand thereof), capped at each end with a structural motif. In this instance, the structural motif may be simple or complex. One of the capped ends is closed if made according to this method, i.e. the vector is made entirely from one strand of nucleic acid, whilst the opposite end is not, formed by the 5′ and 3′ terminal nucleotides of the single strand. It is possible to completely covalently close the vector in an additional step.

The template encodes a single stranded nucleic acid. The template encodes:
(i) a first processing motif, adjacent to
(ii) a first structural motif,
(iii) a single strand of said duplex section,
(iv) a second structural motif, adjacent to
(v) a second processing motif
said processing motif includes a sequence capable of forming a base-paired section including a recognition site for an endonuclease containing a cleavage site, and
said structural motif includes at least one sequence capable of forming intramolecular hydrogen bonds and forming a capped end, wherein optionally either of said first or second (left or right) capped ends includes a structural motif containing a binding motif.

The template may be amplified using rolling circle amplification, producing a single stranded concatemer. This concatemer may be processed into single stranded intermediates using an endonuclease.

The single stranded intermediates may then be contacted with a second polymerase, which is preferable not strand displacing, using the intermediate as a template to extend the 3′ end such that the duplex section is formed.

The strand may be extended as far as the free 5′ end, at which point the vector may be contacted with an enzyme such as a ligase and the nick closed between adjacent residues.

The amplification process or extension process will require the addition of substrates (i.e. appropriate nucleosides for nucleic acid generation), and any co-factors (such as salts, ions or the like). Appropriate conditions for the reaction include the presence of buffers and temperatures at which the enzymes can operate. Appropriate conditions for rolling circle amplification may be isothermal. Appropriate conditions for strand extension may be isothermal.

Amplification is the production of multiple copies of a nucleic acid template, or the production of multiple nucleic acid sequence copies that are complementary to the nucleic acid template. In the methods of the invention, it is preferred that amplification refers to the production of multiple nucleic acid sequence copies that are complementary to the nucleic acid template.

It is preferred, where the template is double stranded, that techniques are used to ensure that the strand complementary to the desired product is used as the template. This may be achieved by several methods discussed further below.

When used in amplification or extension, nucleosides are compounds wherein a nucleic acid base (nucleobase) is linked to a sugar moiety. The nucleic acid base may be a natural or a modified/synthetic nucleobase. The nucleic acid base may include a purine base (e.g., adenine or guanine), a pyrimidine (e.g., cytosine, uracil, or thymine), or a deazapurine base, amongst others. The nucleic acid base may be a ribose or a deoxyribose sugar moiety. The sugar moiety may include a natural sugar, a sugar substitute, a substituted sugar, or a modified sugar. The nucleoside may contain a 2′-hydroxyl, 2′-deoxy, or 2′, 3′-dideoxy forms of the sugar moiety.

Nucleotides or nucleotide bases refer to nucleoside phosphates. This includes natural, synthetic, or modified nucleotides, or a surrogate replacement moiety (e.g., inosine). The nucleoside phosphate may be a nucleoside monophosphate (NMP), a nucleoside diphosphate (NDP) or a nucleoside triphosphate (NTP). The sugar moiety in the nucleoside phosphate may be a pentose sugar, such as ribose. A nucleotide may be, but is not limited to, a deoxyribonucleoside triphosphate (dNTP) or a ribonucleoside triphosphate (rNTP).

Nucleotide analogues are compounds that are structurally similar to naturally occurring nucleotides. The nucleotide analogue may have an altered backbone, sugar moiety, nucleobase, or combinations thereof. It will be understood that the use of such analogues results in nucleic acids which may have different base-pairing properties and the interactions that occur when such bases are stacked may be different to those seen in natural nucleic acids.

The amplification reaction and/or extension reaction is preferably isothermal (at a constant temperature), unlike amplifications such as PCR which require temperature cycling. The methods may be used in the amplification of any appropriate template, preferably a circular nucleic acid template. The nucleic acid template can be provided in any appropriate amount to the reaction, including a minimal amount.

It is preferred that the nucleic acid template is amplified using RCA.

The polymerase enzyme or enzymes used for amplification may be a proofreading or a non-proofreading nucleic acid polymerase. The nucleic acid polymerase used may be a strand displacing nucleic acid polymerase. The nucleic acid polymerase may be a thermophilic or a mesophilic nucleic acid polymerase.

The method may require a highly processive, strand-displacing polymerase to amplify the nucleic acid template under conditions for high fidelity amplification. The ability for the polymerase to accurately replicate the template is referred to as the fidelity of a polymerase. In addition to effective discrimination of correct versus incorrect nucleotide incorporation, some polymerases possess a no 5′ exonuclease activity. This proofreading activity is used to excise incorrectly incorporated bases that are then replaced with the correct one. High-fidelity amplification utilises polymerases that couple low misincorporation rates with proofreading activity to give faithful replication of the template. Alternatively a non-strand displacing enzyme may be used, in conjunction with a helicase.

The amplification reaction may employ a polymerase that generates single stranded, amplified nucleic acid after amplification. The polymerase is therefore capable of strand displacement synthesis.

A Phi29 DNA polymerase or Phi29-like polymerase may be used for amplifying a template in some embodiments. Alternatively, a combination of a Phi29 DNA polymerase and another polymerase may be used.

The amplification reaction may employ a low concentration of primer in one version of the method. The present inventors have found that a low concentration of primer is advantageous, since it enables the amplification reaction to generate only single stranded nucleic acid. A primer is a short linear oligonucleotide which hybridises to a sequence within the template to prime the nucleic acid synthesis reaction. The primer may be any nucleic acid, such as RNA, DNA, non-natural nucleic acid or a mixture of the same. The primer may contain natural, synthetic, or modified nucleotides.

Alternatively, assuming that the template is a double stranded circular template, a nicking enzyme may be employed to make a nick on one strand of the double stranded template. This leaves an entry point for the polymerase, which then utilises the nicked strand of the template itself to prime the nucleic acid synthesis reaction.

The nucleic acid template is therefore amplified by contacting the template with at least a polymerase and nucleotides and incubating the reaction mixture under conditions suitable for nucleic acid amplification. The amplification of the nucleic acid template may be performed under isothermal conditions. Additional components may include one or more of: a nicking enzyme (nickase), a cofactor (e.g. magnesium ions), a primer, a primase, a helicase, and/or a buffering agent.

Rolling circle amplification of a circular template generates a linear single stranded concatemer with adjacent multiple repeats encoded by the template (each one called a sequence unit herein). Due to the nature of the template, this means that each sequence unit includes a section for the formation of a duplex flanked by structural motifs and the outer flanking is by processing motifs. Each sequence unit may also include backbone sequence.

The concatemer may be processed into the nucleic acid constructs using an endonuclease. The cleavage site releases the terminal residue of the structural motif.

When the cleavage site in the concatemeric nucleic acid is cut by the requisite endonuclease, this releases the structural motif from the processing motif, enabling the formation of the capped end under the appropriate conditions.

The amplification and processing reactions may occur simultaneously, i.e. the endonuclease may be present to process the concatemer as soon as it is formed, or there may be a delay in adding the endonuclease until the amplification is further advanced, or indeed complete.

The initial steps of this method prepare a single stranded nucleic acid with capped ends formed by the structural motifs. The structural motif can in some instances provide a proportion of the sequence to form the capped end, such that it is further extended in the second step to form a complete capped end. It may be necessary to contact this single stranded nucleic acid intermediate with a nickase to expose the 3′ nucleotide for extension if it is entirely secured.

The following step of the method is to contact the single stranded nucleic acid intermediate with a polymerase enzyme. The polymerase enzyme extends the free 3′ end of the intermediate and uses the single stranded portion of the “duplex section” as a template to synthesise the complementary sequence for this section, and thus form the duplex. The entire “duplex section” may therefore be created by extension of the strand. The strand may be extended as far as the free 5′ end of the intermediate, leaving just a nick between two adjacent residues. This nick can be closed using an appropriate enzyme such as a ligase, in order to completely covalently close the molecule.

The second step may require a polymerase which is not strand displacing. It may be any suitable polymerase including an RNA polymerase to make a hybrid duplex. Suitable enzymes include Q5® High-Fidelity DNA Polymerase (NEB, US), Q5U® Hot Start High-Fidelity DNA Polymerase (NEB), Phusion® High-Fidelity DNA Polymerase (NEB), OneTaq® DNA Polymerase (NEB), Taq DNA Polymerase (NEB), LongAmp® Taq DNA Polymerase (NEB), Epimark® Hot Start Taq DNA Polymerase (NEB), T7 DNA Polymerase (NEB), DNA Polymerase I (NEB), SP6 RNA Polymerase (NEB), T7 RNA Polymerase (NEB), E. coli Poly(A) Polymerase (NEB), Poly(U) Polymerase (NEB), T3 RNA Polymerase (NEB), E. coli RNA Polymerase Core Enzyme (NEB), E. coli RNA Polymerase Holoenzyme (NEB), or Hi-T7® RNA Polymerase (NEB). Terminal transferases may also be appropriate for use in the method of the invention.

Either the amplification step or the extension step may be carried out in the presence of appropriate nucleotides in order to synthesize the nucleic acid. It is possible to supply either step with modified nucleotides in order to incorporate these into the vector.

The method to make the vectors is therefore elegant and efficient.

Template

In the template (1), a sequence encoding one strand of a duplex (104) is flanked on both sides by a sequence encoding a structural motif (103) and the outer flank is provided by a processing motif (101). The encoded sequence is nested, such that the duplex section is flanked by a structural motif, which in turn is directly adjacent to a processing motif, the structural motif and the processing motif together forming the formatting element. The sequences of the processing motif and the structural motif are thus contiguous. Alternatively put, the formatting element at each end of the duplex section are in the opposite or mirrored orientation, ensuring that the structural motif is closest to the duplex section, whilst the processing motif is the outermost part of the formatting element.

The formatting element is unique, but is not present in complete form in the final product, since the processing motif is cleaved from the structural motif. The action of the endonucleases during processing ensures that the cleavage site of the processing motif is cut, therefore discarding the processing motif. It is thus a mechanism by which to produce a useful product that is partially removed, ensuring that the final product contains the minimum amount of unnecessary sequences, providing more room for the duplex section. Thus, the processing motif and the adjacent structural motif are effectively joined until the cleavage site is cut, releasing the terminal residue of the product. The combination of a processing motif adjacent to a structural motif, effectively separated by a cleavage site for an endonuclease, enables the direct production of a single stranded nucleic acid with sequestered ends from a longer single stranded nucleic acid molecule in a single step process, using an endonuclease. The processing motif is removed from the single stranded nucleic acid via processing with a restriction enzyme, and is not present in the single stranded nucleic acid with sequestered ends.

The formatting element is effectively cleaved by the action of the endonuclease, and therefore partially removed from the final product.

Processing Motif

A processing motif (101) includes sequences capable of forming a base-paired section (201) including a recognition site for an endonuclease and an associated cleavage site. It will be appreciated that the cleavage site can be remote from the recognition site, but that both are generally required to be in a duplexed structure.

In one format, a processing motif may be capable of forming a base-paired section due to the inclusion of at least one region of sequence which is capable of binding to another sequence within the processing motif, these sections may be seen to be self-complementary in sequence. These sequences may be contiguous or may be separated by a spacer element. Such motifs may be designed by including complementary stretches of sequence in the single stranded nucleic acid. It will be appreciated that although both sequences are present on the same strand of nucleic acid, the design of the molecules ensures that one sequence is in the correct orientation to bind to the other, intramolecularly. For example, in DNA, the sequences need to run antiparallel in order for the base pairs to form. Such motifs are common amongst viral single stranded genomes, for example.

The base-paired section of a processing motif may be contiguous, such that the section forms a hairpin or the like. The nucleic acid may form antiparallel double stranded hairpin like structures. The hairpin structure consists of a double stranded base paired region called a stem. Alternatively the base-paired section of a processing motif may include a spacer sequence between the two stretches of sequence capable of base-pairing, such that structures such as stem-loops are formed. The spacer may be any suitable length. The hairpin may be formed of a nucleic acid sequence which is palindromic, as defined herein.

The base paired or double stranded section of the nucleic acid molecule can also have complementary sequence. Base pairing and duplexes are defined further herein.

In the base-paired section of a processing motif, there is included a recognition site for an endonuclease, and an associated cleavage site. It is preferred that the cleavage site forms at the footing of the base-paired section, such that the entire processing motif may be cleaved from the single strand using the requisite endonuclease.

The base-pairing occurs between at least two sections of sequence within the single strand. This base-pairing may be standard (i.e. Watson and Crick classical base pairs which are adenine (A)-thymine (T) in DNA, adenine (A)-uracil (U) in RNA, and cytosine (C)-guanine (G) in both) or non-canonical (i.e. Hoogsteen base pairs or interactions among carbon-hydrogen and oxygen/nitrogen groups and the like). These are described elsewhere.

The template includes one or more sequences encoding a processing motif with any of these characteristics. The processing motifs may be different sequences.

The template may contain a sequence encoding a first processing motif and a sequence encoding a second processing motif. Encoded by the template, the first and second processing motifs are positioned at the outside edge of the structural motif (and within the formatting element), such that each end of the duplex section finishes with formatting elements that are in the opposite orientations (forward and reverse).

Given the nature of the requirements for the processing motif in the single stranded nucleic acid concatemer (prior to processing), the sequence of the first and second processing motifs may be the same or different. If they are the same, then the restriction site forms at the footing of the base-paired section, such that the entire processing motif may be cleaved from the single strand using the requisite endonuclease. Therefore, regardless of the orientation of the processing motif with respect to the duplex section (before or after) then the whole processing motif can be cleaved from the nucleic acid, since the cleavage site is at the footing of the base-paired section, which could also be described as the final base pair of the paired section, or the base thereof.

Alternatively, the first and second processing motifs in the single stranded nucleic acid concatemer (prior to processing) may be different, such that each recognition site for an endonuclease containing a cleavage site is also different, enabling the use of different endonucleases when processing the single stranded concatemer of the invention.

The template may therefore include sequences encoding identical or different first and second processing motifs.

An endonuclease is an enzyme, whether proteinaceous or composed of nucleic acid such as DNA, that cleaves a phosphodiester bond within a polynucleotide chain. In this invention, a cut through double-stranded nucleic acid is required in order to produce the nucleic acid molecule with sequestered ends. Therefore, a combination of two endonucleases may be required, each one cutting through a single strand. Alternatively, a single enzyme that cleaves both strands may be employed. The endonuclease may be a nicking endonuclease, a homing endonuclease, a guided endonuclease such as Cas9, or a restriction endonuclease, for example. A nicking endonuclease may be a modified restriction endonuclease that has been modified to cut only one strand.

In one aspect, the endonuclease is a restriction endonuclease.

A restriction endonuclease is an enzyme that cleaves double stranded nucleic acid at cleavage sites within or near to a specific recognition site. To cut, all restriction endonucleases make two incisions, once through each backbone (i.e. each strand) of the duplex. Since a restriction endonuclease requires the presence of double stranded nucleic acid in order to recognise the recognition site, such a structure is required in order to allow the endonuclease to cleave the nucleic acid. Therefore, the present inventors propose the construction of a base-paired section within the single stranded nucleic acid, preferably using self-complementary sequences, such that the single stranded molecule forms a double stranded structure including the recognition and cleavage sites.

Restriction endonucleases recognize a specific sequence of nucleotides and produce a double-stranded cut in the duplex. The recognition site can also be classified by the number of bases, usually between 4 and 8 bases. Many, but not all, of the recognition sites are palindromic, and this property is very useful when designing the processing motif, since it aids the design of the sequence enabling it to be placed in a base-paired section more easily. In the single stranded format, the sections that are capable of forming the palindrome when base-paired to each other are called inverted repeat sequences. These two sequences may be separated by a spacer sequence in the single stranded nucleic acid.

The restriction endonuclease may be a blunt cutter (i.e. cut straight through the base-paired section) or cut in an offset fashion (i.e. cut is staggered through the base-paired section). The cleavage site can be within the recognition site, or nearby, and thus the cleavage site does not need to be part of the recognition site. Therefore, the cleavage site is associated with the recognition site, but does not necessarily form part of it.

Many thousands of restriction endonucleases are known, both natural and engineered, together with their recognition and cleavage sites. Any suitable recognition and cleavage sites may be included in a processing motif. Exemplary restriction endonucleases commonly used in cloning and the like are HhaI, HindIII, NotI, EcoRI, ClaI, BamHI, BglII, DraI, EcoRV, PstI, SalI, SmaI, SchI and XmaI. Many are commercially available from suppliers such as New England Biolabs and ThermoFisher Scientific.

In order for the cleavage using the endonuclease to release the structural motif from the formatting element in the single stranded nucleic acid concatemer, it is preferred that the cleavage site is adjacent to the structural motif in the template, such that the terminal nucleotide of the structural motif forms the terminal residue and end of the single stranded nucleic acid molecule intermediate.

Within the template, encoded is a formatting element, one part of which is a sequence encoding a structural motif, which is designed to be folded in the intermediate single stranded nucleic acid molecule and the final vector. The structural motif may secure the ends (i.e. 5′ and 3′ ends for DNA and RNA) of the single stranded nucleic acid molecule intermediate, such that the 3′ and 5′ ends may ultimately be joined, in particular, such that the 3′ end acts as a primer for extension.

Structural Motif

A structural motif (103) includes sequences (105 and 106) capable of forming a base paired section or duplex internally. This base-paired section or duplex may form in the concatemer prior to processing with an endonuclease, or it may form after processing with an endonuclease, once the processing motif has been removed from the concatemer. These structures may not form until the processing motif has been cleaved by the endonuclease.

The duplex may be formed by base-pairing between at least two sections of sequence within the single strand. This base-pairing may be standard (i.e. Watson and Crick classical base pairs which are adenine (A)-thymine (T) in DNA, adenine (A)-uracil (U) in RNA, and cytosine (C)-guanine (G) in both) or non-canonical (i.e. Hoogsteen base pairs, interactions among carbon-hydrogen and oxygen/nitrogen groups and the like). Hoogsteen pairs allow formation of particular structures of single stranded nucleic acid G-rich segments called G-quadruplexes, or C-rich segments called i-motifs. G quadruplexes generally require four triplets of G, separated by short spacers. This permits assembly of planar quartets which are composed of stacked associations of Hoogsteen bonded guanine molecules.

A structural motif may therefore include sections of sequence which are self-complementary or complementary to another sequence within single stranded nucleic acid molecule, i.e. to the duplex section or a spacer sequence within the duplex section.

A structural motif may include sequences for forming more than one base-paired section or duplex, each of which are separated by spacer sequences of single stranded nucleic acid, or the base paired sections or duplexes may form part of larger structures which may include any one or more of the following: hairpin; single stranded regions; bulge loop; internal loop; multi-branched loop or junction. The structural motif may be as described above in relation to the vector. The structural motif may include a binding motif, which is also as described above in relation to the vector.

Once the structural motif has formed at least one base-paired section or duplex, the terminal residue of the single stranded nucleic acid molecule may be secured. The terminal nucleotide (or residue) at either end of the single stranded DNA is preferably base paired to another residue in the intermediate. This renders the terminal residues suitable for extension with a polymerase enzyme or ligating to an extended strand.

It is preferred that the terminal end (terminal nucleotide) is not in single stranded form in the single stranded nucleic acid intermediate. These ends are stabilised by presence of base pairing between each terminal residue and another part of the single stranded nucleic acid intermediate.

A structural motif from the concatemeric nucleic acid molecule, once processed, forms one end of the single stranded nucleic acid construct. The terminal residue is generally secured by the structural motif.

Preferred structural motifs according to the present invention include sequences which can fold as hairpins, stem loops, junctions, pseudoknots, ITRs, modified ITRs, synthetic ITRs, i-motifs and G-quadruplexes. The structural motifs may be as hereinbefore described.

A hairpin is a structure in a nucleic acid, such as DNA or RNA, formed due to base-pairing between neighbouring complementary sequences of a single strand of the nucleic acid. The neighbouring complementary sequences may be separated by a few nucleotides, e.g. 1-10 or 1-5 nucleotides. If a loop of non-complementary sequence is included between the two sections of complementary sequence, this forms a hairpin loop or a stem loop. The loop may be of any suitable length, as may the stem or double stranded section. Other similar structures include lariats.

The structural motifs at each end can fold into the same particular structure (i.e. a hairpin, stem loop, ITR or the like) or they can each independently be designed to fold into different structures (i.e. the first end is a hairpin and the second end is an ITR).

As discussed previously, the structural motifs can include binding motifs, as hereinbefore described. They can form functional structures such as aptamers and the like. In the exemplified process, the two complementary sequences (105 and 106) flank a binding motif within the structural motif. Such a design permits the inventors to remove the central section for the binding motif and replace it with an alternative motif whilst ensuring that the structural motif is suitable for use in the method of the invention, since the flanking complementary sequences ensure the formation of a stem structure to “support” the binding motif.

Duplex

The template also encodes for a single strand of the duplex section. The duplex section can be any desired nucleic acid sequence, of any suitable length.

The duplex section preferably includes a gene or fragment thereof, optionally within an expression cassette. The duplex section may include a transgene, such as a gene or genetic material, for expression in a cell. The transgene may be operably connected to a promoter sequence within an expression cassette.

The duplex section may include a sequence which encodes a therapeutic product. The therapeutic product may be a DNA aptamer, a protein, a peptide, or an RNA molecule, such as small interfering RNA. In order to provide for therapeutic utility, such a duplex section may comprise an expression cassette comprising one or more promoter or enhancer elements and a gene or other coding sequence which encodes an mRNA or protein of interest. The expression cassette may comprise a eukaryotic promoter operably linked to a sequence encoding a protein of interest, and optionally an enhancer and/or a eukaryotic transcription termination sequence.

The duplex section may be used for production of DNA for expression in a host cell, particularly for production of DNA vaccines. DNA vaccines typically encode a modified form of an infectious organism's DNA, such as the entire genome. DNA vaccines are administered to a subject where they then express the selected protein of the infectious organism, initiating an immune response against that protein which is typically protective. DNA vaccines may also encode a tumour antigen in a cancer immunotherapy approach. Any DNA vaccine may be used as the duplex section.

Also, the process of the invention may produce other types of therapeutic DNA molecules e.g. those used in gene therapy. For example, such DNA molecules can be used to express a functional gene where a subject has a genetic disorder caused by a dysfunctional version of that gene. Examples of such diseases are well known in the art.

It is preferred that the portion of the template encoding the duplex section or the structural motif lacks a bacterial origin of replication, lacks resistance genes (i.e. for antibiotics), lacks prokaryotic patterns of methylation (except for DNA vaccines where the same may be helpful), or any other marker of foreign DNA. These entities can, however, be present outside the duplex section and structural motif, since the rest of the template is processed and removed from the product.

The template is preferably circular or capable of circularisation. The template may be double stranded or single stranded.

If the template is double stranded, it is preferred that it includes a sequence for a nicking enzyme prior to the first processing motif. Alternatively known as nicking endonucleases, these enzymes hydrolyse only one strand of the duplex, to produce nucleic acid molecules that are “nicked”, rather than cleaved. This provides a start-point for rolling circle amplification without the need for additional primer and can ensure that only one strand of nucleic acid concatemer is produced in the amplification reaction. Such enzymes are commercially available, for example from New England Biolabs and Thermo Fisher Scientific. These enzymes are specific enough such that a recognition and cleavage site can be designed on the relevant strand of the template to ensure the correct strand is used directly as the template.

The template may be any suitable nucleic acid, either natural such as DNA or RNA, or artificial as discussed previously. It is preferred that the template is DNA.

The nucleic acid produced may be any suitable nucleic acid such as DNA, RNA or a hybrid thereof.

Preferred are DNA vectors. The vector may include modified bases, such that other entities may be connected to the vector using simple chemical means.

Amplification of the Template

In order to produce the single stranded nucleic acid intermediates, the template has to be amplified enzymatically.

The template may be amplified with one or more polymerase enzymes. The polymerase enzyme can use the template to synthesise a complementary nucleic acid copy, if provided with sufficient raw materials or substrates (such as nucleotides) and co-factors (such as metal ions and the like) in order to amplify the nucleic acid.

Any suitable polymerase enzyme may be used for this amplification step, and it is possible to use one enzyme, or a combination of enzymes.

The enzyme may be a DNA polymerase or RNA polymerase depending on the nature of the template, or an artificial, modified, engineered or mutant polymerase in order to use a synthetic template or to manufacture a synthetic single stranded nucleic acid.

Amplification is preferred to proceed via strand displacement methods. This is an isothermal method that does not require repeated cycles of heating and cooling (as PCR does), but the polymerase enzyme is capable of displacing any strand which is annealed to the template. Strand-displacement type polymerases are known, including Phi29, Deep Vent®, BST DNA polymerase I and variants of the same. This means that multiple polymerases can act on the same template at the same time, each one displacing the nascent strand produced by the earlier polymerase.

The most preferred strand displacement amplification technique is rolling circle amplification (RCA). In this method of amplification, strand displacing polymerases progress continually around a circular template whilst extending the nascent oligonucleotide. This leads to the generation of long concatemeric strands of nucleic acid.

It is preferred that the amplification reaction is allowed to initiate on a double stranded circular template by nicking the template with a nicking endonuclease. Such enzymes are discussed above. By nicking a single strand of a double stranded template, this opens up the template for the polymerase to bind, and it may utilise the free 3′ end created to extend this strand into a concatemeric nucleic acid by processing around the circular template many times.

The use of a nicking site in the template and a nicking endonuclease also permits the method only to make a single stranded concatemer from the RCA, and prevents the amplification of the opposite strand, since only one backbone is cleaved using the enzyme.

Thus, the use of a nicking site in the template is preferred, since it allows for the production of the desired product, and prevents the unwanted amplification of the complementary strand of a double stranded template.

Alternatively, the present inventors have found that using a very low quantity of a specific primer which is designed to anneal to the desired template strand (and not its complementary strand), that the amplification can be forced to proceed to make large quantities of only one strand of a double stranded template. In this aspect, only picomolar quantities of primer are required. Thus, the primer may be supplied in a quantity of 1 pM to 100 nM.

If the template is single stranded, then it is possible to use a primer to initiate the rolling circle amplification. Preferably, the primer is designed only to anneal to the template and not to the concatemeric nucleic acid molecule, thus ensuring that only one species of concatemer is made.

The template is contacted with at least one polymerase. One, two, three, four or five different polymerases may be used. The polymerase may be any suitable polymerase, such that it synthesises polymers of nucleic acid. The polymerase may be a DNA or RNA polymerase. Any polymerase may be used, including any commercially available polymerase. Two, three, four, five or more different polymerases may be used, for example one which provides a proofreading function and one or more others which do not. Polymerases having different mechanisms may be used e.g. strand displacement type polymerases and polymerases replicating nucleic acid by other methods. A suitable example of a DNA polymerase that does not have strand displacement activity is T4 DNA polymerase.

A polymerase may be highly stable, such that its activity is not substantially reduced by prolonged incubation under process conditions. Therefore, the enzyme preferably has a long half-life under a range of process conditions including but not limited to temperature and pH. It is also preferred that a polymerase has one or more characteristics suitable for a manufacturing process. The polymerase preferably has high fidelity, for example through having proofreading activity. Furthermore, it is preferred that a polymerase displays high processivity, high strand-displacement activity and a low Km for nucleotides and nucleic acid. A polymerase may be capable of using circular and/or linear DNA as template. The polymerase may be capable of using double stranded or single stranded nucleic acid as a template. It is preferred that a polymerase does not display exonuclease activity that is not related to its proofreading activity.

The skilled person can determine whether or not a given polymerase displays characteristics as defined above by comparison with the properties displayed by commercially available polymerases, e.g. Phi29 (New England Biolabs, Inc., Ipswich, MA, US), Deep Vent® (New England Biolabs, Inc.), Bacillus stearothermophilus (Bst) DNA polymerase I (New England Biolabs, Inc.), Klenow fragment of DNA polymerase I (New England Biolabs, Inc.), M-MuLV reverse transcriptase (New England Biolabs, Inc.), VentR® (exo-minus) DNA polymerase (New England Biolabs, Inc.), VentR® DNA polymerase (New England Biolabs, Inc.), Deep Vent® (exo-) DNA polymerase (New England Biolabs, Inc.), Bst DNA polymerase large fragment (New England Biolabs, Inc.), hi-fidelity fusion DNA polymerase (e.g., Pyrococcus-like, New England Biolabs, MA), Pfu DNA polymerase from Pyrococcus furiosus (Agilent, La Jolla, CA), Sequenase™ variant of T7 DNA polymerase, T7 DNA polymerase, T4 DNA polymerase, DNA polymerase from Pyrococcus species GB-D (New England Biolabs, MA), or DNA polymerase from Thermococcus litoralis (New England Biolabs—NEB, MA).

Alternatively, the polymerase may be a DNA-dependent RNA polymerase. Exemplary enzymes include T3 RNA Polymerase, T7 RNA Polymerase, Hi-T7™ RNA Polymerase, SP6 RNA Polymerase, E. coli Poly(A) Polymerase, E. coli RNA Polymerase, and E. coli RNA Polymerase, Holoenzyme (all available from NEB).

Where a high processivity is referred to, this typically denotes the average number of nucleotides added by a polymerase enzyme per association/dissociation with the template, i.e. the length of primer extension obtained from a single association event.

Strand displacement-type polymerases are preferred. Preferred strand displacement-type polymerases are Phi29, Deep Vent and Bst DNA polymerase I or variants of any thereof. “Strand displacement” describes the ability of a polymerase to displace complementary strands on encountering a region of double stranded DNA during synthesis. The template is thus amplified by displacing complementary strands and synthesizing a new complementary strand. Thus, during strand displacement replication, a newly replicated strand will be displaced to make way for the polymerase to replicate a further complementary strand. The amplification reaction initiates when a primer or the free end of a single stranded template anneals to a complementary sequence on a template (both are priming events). When nucleic acid synthesis proceeds and if it encounters a further primer or other strand annealed to the template, the polymerase displaces this and continues its strand elongation. It should be understood that strand displacement amplification methods differ from PCR-based methods in that cycles of denaturation are not essential for efficient amplification, as double-stranded template is not an obstacle to continued synthesis of new strands. Strand displacement amplification may only require one initial round of heating, to denature the initial template if it is double stranded, to allow the primer to anneal to the primer binding site if used. Following this, the amplification may be described as isothermal, since no further heating or cooling is required. In contrast, PCR methods require cycles of denaturation (i.e. elevating temperature to 94 degrees Celsius or above) during the amplification process to melt double-stranded DNA and provide new single stranded templates. During strand displacement, the polymerase will displace strands of already synthesised nucleic acid.

A strand displacement polymerase used in the process of the invention preferably has a processivity of at least 20 kb, more preferably, at least 30 kb, at least 50 kb, or at least 70 kb or greater. In one embodiment, the strand displacement DNA polymerase has a processivity that is comparable to, or greater than phi29 DNA polymerase.

The contacting of the template with the polymerase and either a nickase or a primer may take place under conditions promoting annealing of primers to the template. The conditions include the presence of single-stranded DNA allowing for hybridisation of the primers. The conditions also include a temperature and buffer allowing for annealing of the primer to the template. Appropriate annealing/hybridisation conditions may be selected depending on the nature of the primer. An example of preferred annealing conditions used in the present invention include a buffer comprising 30 mM Tris-HCl pH 7.5, 20 mM KCl, 8 mM MgCl₂. The annealing may be carried out following denaturation using heat by gradual cooling to the desired reaction temperature.

The template and polymerase are also contacted with nucleotides. The combination of template, polymerase and nucleotides forms a reaction mixture. The reaction mixture may also comprise one or more primers or alternatively a nicking enzyme (nickase) or a priming enzyme (primase). The reaction mixture may independently also include one or more metal cations or any other required co-factors for nucleic acid synthesis.

A nucleotide is a monomer, or single unit, of nucleic acids, and nucleotides are composed of a nitrogenous base, a five-carbon sugar (ribose or deoxyribose), and at least one phosphate group. Any suitable nucleotide may be used.

The nucleotides may be present as free acids, their salts or chelates, or a mixture of free acids and/or salts or chelates.

The nucleotides may be present as monovalent metal ion nucleotide salts or divalent metal ion nucleotide salts.

The nitrogenous base may be adenine (A), guanine (G), thymine (T), cytosine (C), and/or uracil (U). The nitrogenous base may also be modified bases, such as 5-methylcytosine (m5C), pseudouridine (ψ), dihydrouridine (D), inosine (I), and/or 7-methylguanosine (m7G).

It is preferred that the five-carbon sugar is a deoxyribose, such that the nucleotide is a deoxynucleotide.

The nucleotide may be in the form of deoxynucleoside triphosphate, denoted dNTP. This is a preferred embodiment of the present invention. Suitable dNTPs may include dATP (deoxyadenosine triphosphate), dGTP (deoxyguanosine triphosphate), dTTP (deoxythymidine triphosphate), dUTP (deoxyuridine triphosphate), dCTP (deoxycytidine triphosphate), dTTP (deoxyinosine triphosphate), dXTP (deoxyxanthosine triphosphate), and derivatives and modified versions thereof. It is preferred that the dNTPs comprise one or more of dATP, dGTP, dTTP or dCTP, or modified versions or derivatives thereof. It is preferred to use a mixture of dATP, dGTP, dTTP and dCTP or modified version thereof.

The nucleotides may be in solution or provided in lyophilised form. A solution of nucleotides is preferred.

The nucleotides may be provided in a mixture of one or more suitable bases, including any newly designed artificial bases, preferably, one or more of adenine (A), guanine (G), thymine (T), cytosine (C). Two, three or preferably all four nucleotides (A, G, T, and C) are used in the process to synthesise the nucleic acid.

Concatemer

The concatemer is a nucleic acid molecule with repeated units of the sequence unit present in the template. Each sequence unit includes a sequence for the duplex section flanked on both sides by formatting elements, as described previously. The sequence unit may also include backbone sequence encoded by the template, which is ultimately not present in the vector of the invention.

Concatemeric nucleic acid molecules may comprise multiple sequence units, for example, 10, 50, 100, 200, 500 or even 1000 or more sequence units in continuous series. Concatemeric molecules may be at least 5 kb in size, at least 50 kb, at least 100 kb, or even up to 200 kb in length.

Processing the Concatemeric Nucleic Acid Molecule

Once the template has been amplified, or even during amplification, the concatemeric nucleic acid may be processed into single stranded intermediates using the requisite endonucleases which will cleave the one or more processing sites.

It is therefore preferred that the processing motif is capable of forming a base-paired portion whilst in the form of a concatemeric nucleic acid. Thus, the processing motif may be designed such that the base pairs form under the conditions suitable for isothermal amplification. Once these base-paired portions have formed within the concatemeric nucleic acid, recognition sites for the endonucleases form, together with the necessary cleavage sites. This elegant system allows for the processing of the concatemer, despite the fact that it is only a single strand of nucleic acid. It is the design of the template that allows for the formation of processing sites within the concatemeric nucleic acid, allowing for a single step to process this concatemer by the addition of one or more endonucleases.

The endonucleases may be added once the amplification reaction is complete, whilst it is underway or at the start of the amplification reaction. It is preferred that the amplification reaction is underway before the endonucleases are added, to ensure that the concatemeric nucleic acid is processed quickly. Alternatively, the amplification process may be allowed to complete (i.e. template exhausted, nucleotides exhausted, reaction mixture too viscous) prior to the addition of endonucleases.

Also produced are side products that consist of the processing motif plus any associated template “backbone”.

Extending the 3′ End and Optional Closure

The 3′ end of the single stranded intermediate following cleavage with the endonuclease is base paired to the intermediate such that it is capable of acting as a primer. It is possible to design a nicking site at or near the 3′ end to make sure that the 3′ end is available for extension following application of a nickase.

The intermediate is contacted with one or more polymerase enzymes. One, two, three, four or five different polymerases may be used. The polymerase may be any suitable polymerase, such that it synthesises polymers of nucleic acid. The polymerase may be a DNA or RNA polymerase. Any polymerase may be used, including any commercially available polymerase. Two, three, four, five or more different polymerases may be used, for example one which provides a proofreading function and one or more others which do not. Polymerases having different mechanisms may be used, but it is preferred that the polymerase does not strand displace. A suitable example of a DNA polymerase that does not have strand displacement activity is T4 DNA polymerase.

A polymerase may be highly stable, such that its activity is not substantially reduced by prolonged incubation under process conditions. Therefore, the enzyme preferably has a long half-life under a range of process conditions including but not limited to temperature and pH. It is also preferred that a polymerase has one or more characteristics suitable for a manufacturing process. The polymerase preferably has high fidelity, for example through having proofreading activity. Furthermore, it is preferred that a polymerase displays high processivity, and a low Km for nucleotides and nucleic acid. A polymerase may be capable of using linear DNA as template. The polymerase may be capable of using single stranded nucleic acid as a template. It is preferred that a polymerase does not display exonuclease activity that is not related to its proofreading activity.

The skilled person can determine whether or not a given polymerase displays characteristics as defined above by comparison with the properties displayed by commercially available polymerases, e.g. Deep Vent® (New England Biolabs, Inc.), Bacillus stearothermophilus (Bst) DNA polymerase I (New England Biolabs, Inc.), Klenow fragment of DNA polymerase I (New England Biolabs, Inc.), M-MuLV reverse transcriptase (New England Biolabs, Inc.), VentR® (exo-minus) DNA polymerase (New England Biolabs, Inc.), VentR® DNA polymerase (New England Biolabs, Inc.), Deep Vent® (exo-) DNA polymerase (New England Biolabs, Inc.), Bst DNA polymerase large fragment (New England Biolabs, Inc.), hi-fidelity fusion DNA polymerase (e.g., Pyrococcus-like, New England Biolabs, MA), Pfu DNA polymerase from Pyrococcus furiosus (Agilent, La Jolla, CA), Sequenase™ variant of T7 DNA polymerase, T7 DNA polymerase, T4 DNA polymerase, DNA polymerase from Pyrococcus species GB-D (New England Biolabs, MA), or DNA polymerase from Thermococcus litoralis (New England Biolabs, MA).

Alternatively, the polymerase may be a DNA-dependent RNA polymerase. Exemplary enzymes include T3 RNA Polymerase, T7 RNA Polymerase, Hi-T7™ RNA Polymerase, SP6 RNA Polymerase, E. coli Poly(A) Polymerase, E. coli RNA Polymerase, and E. coli RNA Polymerase, Holoenzyme (all available from NEB).

Where a high processivity is referred to, this typically denotes the average number of nucleotides added by a polymerase enzyme per association/dissociation with the template, i.e. the length of primer extension obtained from a single association event.

The intermediate and the polymerase enzyme will also be placed under suitable conditions and with suitable reagents in order to extend the 3′ end of the intermediate using the single strand of the duplex section as a primer to synthesize the complementary strand of the duplex. Thus, the action of the polymerase in this step is to synthesize the duplex using the single stranded section as a template. It is preferred that the 3′ end is extended as far as the end of the duplex section, most preferably to adjacent to the 5′ end of the intermediate. Extending the 3′ end until it is adjacent to the 5′ end is advantageous, since it permits the covalent closure of the vector using an enzyme such as a ligase. Suitable conditions for the extension reaction are described in relation to the amplification step, including the provision of reagents.

The 3′ and 5′ ends may be ligated, closing the vector covalently. The extended intermediate may be contacted with a ligase enzyme in order for the vector to be covalently closed. The ends of the DNA vector are joined together by the formation of phosphodiester bonds between the 3′-hydroxyl of one end with the 5′-phosphoryl of another. RNA may also be ligated similarly. A co-factor is generally involved in the reaction, and this is usually ATP or NAD⁺.

Nuclear Targeting

Histones are the most abundant proteins in the nucleus and a distinct import pathway is involved in an active transport of histones from the cytoplasm to the nucleus. This fact means that hijacking the histone import pathway is a universal method to enhance nuclear localization of DNA of interest for therapeutic or other purposes.

Nucleolin is a shuttling protein with diverse functions and whilst it can be found in different cellular compartments, its greatest abundance is in the nucleus.

Human telomeres are maintained by multiple proteins and some of them bind specifically to G-quadruplex structures that can be formed by repeats of telomere sequence (TTAGGG)n, while four repeats (TTAGGG)4 constitute the minimal sequence necessary to form G-quadruplex structure. Moreover, there is an evidence showing that ends of chromosomes that are comprised of repeats (TTAGGG)n effectively bind DNA aptamers that are selective to G-quadruplex structures.

The minimal sequence of DNA that is necessary to form G-quadruplex structure (TTAGGG)4 has been employed by the inventors to construct a structural motif which is capable of forming a structure with a binding motif that can hijack nuclear import of proteins that natively recognize G-quadruplex structures.

The structural motifs exemplified herein target three distinct classes of protein factors to hijack their nuclear import pathways. Closing one or both ends of duplex DNA of interest with a cap which includes a binding motif constitutes an innovative strategy to enhance nuclear uptake and expression of DNA of interest for therapeutic or other purposes.

The invention will now be described with reference to the following non-limiting examples.

### Example 1

Nuclear Import Enhanced by the Presence of Binding Motifs in the DNA Vector

Vector Synthesis:

Vector DNA with the secreted embryonic alkaline phosphatase (SEAP) gene was synthesized in house. Various versions were constructed, each version with a different capped end. These capped ends were:
i) histone H4 aptamer,
ii) nucleolin aptamer,
iii) telomere G-quadruplex structure.

Various linear duplexed DNA with a mammalian expression cassette [promoter—gene—polyA sequence] [Ef1α-SEAP-SV40poly(A)] was generated, each version contained the structural motif for the various capped ends, each of which was placed downstream of the expression cassette and therefore formed the right capped end of the vector.
i) histone H4 (H4_Gq, and H4_sl)
ii) nucleolin: (nucl)
iii) human Telomere G-quadruplex (hTel)

The left end of the vector was also capped with a structural motif, in this case a sequence for a simple stem-loop with a 3-nucleotide loop GAA, which has no binding affinity to known protein factors.

The reference DNA (referred to a “no aptamer”) contained the 3-nucleotide GAA loop at both ends.

Transfection:

DNA was transfected to HEK293 cells (ATCC) using commercially available PElpro transfection reagent (Polyplus-transfection) following the manufacturer's guidelines.

Briefly, the cells were seeded in a 6-well plate 24 h before the transfection at the density of 7×10⁵ cells per well, in a total volume of 2 ml DMEM culture medium supplemented with 10% FBS (Sigma), 2 mM L-glutamine (Sigma) and 1% non-essential amino acid solution (Sigma).

The cells were incubated at 37° C. in a 5% CO₂ incubator until they reached 70% confluency (24 h). Vector complexes with PElpro were generated as shown in table 6. Upon incubation for 15 min at room temperature, the DNA-PEI complexes in serum-free DMEM were added to the cell culture (dropwise). The plates with cell cultures were placed in an incubator maintaining in 5% CO₂ atmosphere at 37° C. Media was collected after 9 hours for secreted alkaline phosphatase (SEAP) activity, and biological duplicates were performed for all samples. Luminescence-based SEAP Reporter Gene Assay Kit (Abcam) was used to determine levels of SEAP expression (U/ml of media).

To ensure equal transfection efficiency across the experiment, a co-transfection with CMV-eGFP vector was performed (10% of the DNA mass used for transfection was linear DNA encoding eGFP). Median fluorescence was measured after 48 h using flow cytometry and confirmed to be invariant across the experiment.

TABLE 6
Transfection protocol:
DNA dilution in media (Mix A)

| Conc. | | Volume | Total |
|---|---|---|---|
| (μg/mL) | DNA construct | (μl) | DNA (μg) |
| 125 | eGFP standard | 2 | 0.25 |
| 125 | Vectors with | 18 | 2.25 |
| | binding motifs | | |
| | and SEAP | | |
| | DMEM media | 230 | |
| | total | 250 | 2.50 |

PEIpro dilution in media (Mix B)

| Solution | Volume (μl) |
|---|---|
| PEIpro | 10 |
| DMEM media | 240 |
| Total | 250 |

Transfection mix (for 2.5 wells)

| Solution | Volume (μl) |
|---|---|
| Mix A (DNA) | 250 |
| Mix B (PEIpro) | 250 |
| Total | 500 |

Add to each well

| Volume (μl) | Total DNA (μg) |
|---|---|
| 200 | 1.00 |

Below are the sequences of the various structural motifs located downstream of the expression cassette (Efla-SEAP-SV40poly(A)). The sequences marked with an underscore show the section of the structural motif that acts as the binding motif (or the section that forms the trinucleotide loop in the control). On each side (flanking) of the binding motif are sequences from the structural motif that in this instance are the complementary sequences that hybridize to each other to form a supporting stem structure, effectively holding the aptamer in place in the vector. Thus, it can be seen that it is possible to design a “template” structural motif into which a specific binding motif may be placed. In this instance, the flanking sequences are complementary to each other.

>no aptamer (control)
ctgctcacctgccagctacggacgcggaacgcgtccgtagctggcaggtgagcag
>H4_Gq
ctgctcacctgccagctacggacgcgtggtggggttcccgggagggcggctacgggttccgtaatcagatttgtgtcgcgtccgtagctggcagg
tgagcag
>H4_SL
ctgctcacctgccagctacggacgcgcgcaggttaaatcccaaatggtccgagggttgcgcgcgtccgtagctggcaggtgagcag
>nucl
ctgctcacctgccagctacggacgcgtggtggtggtggttgtggtggtggtgggcgcgtccgtagctggcaggtgagcag
>hTEL
ctgcgcgctcgctcgctcactgaggcctttagggttagggttagggttagggttggcctcagtgagcgagcgagcgcgcag
Sequence of the EF1α-SEAP-SV40pA cassette
ggctccggtgcccgtcagtgggcagagcgcacatcgcccacagtccccgagaagttggggggaggggtcggcaattgaaccggtgcctagaga
aggtggcgcggggtaaactgggaaagtgatgtcgtgtactggctccgcctttttcccgagggtgggggagaaccgtatataagtgcagtagtcgc
cgtgaacgttctttttcgcaacgggtttgccgccagaacacaggtaagtgccgtgtgtggttcccgcgggcctggcctctttacgggttatggccct
tgcgtgccttgaattacttccacctggctgcagtacgtgattcttgatcccgagcttcgggttggaagtgggtgggagagttcgaggccttgcgctt
aaggagccccttcgcctcgtgcttgagttgaggcctggcctgggcgctggggccgccgcgtgcgaatctggtggcaccttcgcgcctgtctcgctg
ctttcgataagtctctagccatttaaaatttttgatgacctgctgcgacgctttttttctggcaagatagtcttgtaaatgcgggccaagatctgca
cactggtatttcggtttttggggccgcgggggcgacggggcccgtgcgtcccagcgcacatgttcggcgaggcggggcctgcgagcgcggccac
cgagaatcggacgggggtagtctcaagctggccggcctgctctggtgcctggtctcgcgccgccgtgtatcgccccgccctgggcggcaaggctg
gcccggtcggcaccagttgcgtgagcggaaagatggccgcttcccggccctgctgcagggagctcaaaatggaggacgcggcgctcgggagag
cgggcgggtgagtcacccacacaaaggaaaagggcctttccgtcctcagccgtcgcttcatgtgactccacggagtaccgggcgccgtccaggc
acctcgattagttctcgagcttttggagtacgtcgtctttaggttggggggaggggttttatgcgatggagtttccccacactgagtgggtggagac
tgaagttaggccagcttggcacttgatgtaattctccttggaatttgccctttttgagtttggatcttggttcattctcaagcctcagacagtggtt
caaagtttttttcttccatttcaggtgtcgtgacctaggaagcttgccaccatggttctggggccctgcatgctgctgctgctgctgctgctgggcc
tgaggctacagctctccctgggcatcatcccagttgaggaggagaacccggacttctggaaccgcgaggcagccgaggccctgggtgccgccaaga
agctgcagcctgcacagacagccgccaagaacctcatcatcttcctgggcgatgggatgggggtgtctacggtgacagcagccaggatcctaaa
agggcagaagaaggacaaactggggcctgagatacccctggctatggaccgcttcccatatgtggctctgtccaagacatacaatgtagacaa
acatgtgccagacagtggagccacagccacggcctacctgtgcggggtcaagggcaacttccagaccattggcttgagtgcagccgcccgcttt
aaccagtgcaacacgacacgcggcaacgaggtcatctccgtgatgaatcgggccaagaaagcagggaagtcagtgggagtggtaaccaccac
acgagtgcagcacgcctcgccagccggcacctacgcccacacggtgaaccgcaactggtactcggacgccgacgtgcctgcctcggcccgcca
ggaggggtgccaggacatcgctacgcagctcatctccaacatggacattgatgtgatcctgggtggaggccgaaagtacatgtttcgcatggga
accccagaccctgagtacccagatgactacagccaaggtgggaccaggctggacgggaagaatctggtgcaggaatggctggcgaagcgcca
gggtgcccggtatgtgtggaaccgcactgagctcatgcaggcttccctggacccgtctgtgacccatctcatgggcctctttgagcctggagacat
gaaatacgagatccaccgagactccacactggacccctccctgatggagatgacagaggctgccctgcgcctgctgagcaggaacccccgcgg
cttcttcctcttcgtggagggtggtcgcatcgaccacggtcatcacgaaagcagggcttaccgggcactgactgagacgatcatgttcgacgacg
ccattgagaggggggccagctcaccagcgaggaggacacgctgagcctcgtcactgccgaccactcccacgttttctccttcggaggctacccc
ctgcgagggagctccatcttcgggctggcccctggcaaggcacgggacaggaaggcctacacggtcctcctatacggaaacggtccaggctatg
tgctcaaggacggcgcccggccggatgttaccgagagcgagagcgggagccccgagtatcggcagcagtcagcagtgcccctggacgaagag
acgcacgcaggcgaggacgtggcggtgttcgcgcgcggcccgcaggcgcacctggttcacggcgtgcaggagcagaccttcatagcgcacgtc
atggccttcgccgcctgcctggagccctacaccgcctgcgacctggcgccccccgccggcaccaccgacgccgcgcacccagggcggtcccggt
ccaagcgtctggattgagaattcgcccgggcagacatgataagatacattgatgagtttggacaaaccacaactagaatgcagtgaaaaaaat
gctttatttgtgaaatttgtgatgctattgctttatttgtaaccattataagctgcaataaacaagttaacaacaacaattgcattcattttatgtt
tcaggttcagggggaggtgtgggaggttttttaaagcaagtaaaacctctacaaatgtggta

Results

Vectors that contained an aptamer in a capped end to improve nuclear import pathway showed significant increase of SEAP expression in comparison with the control vector (no apt) (FIG. 1). The “vector” minus SEAP showed no expression.

### Example 2

Nuclear Import of a Vector Enhanced by the Presence of a Capped End Including an Aptamer when Reduced Amounts of Vector are Used.

The vectors as described above were transfected to HEK293 using PEI Pro following the manufacturer's protocol, as outlined in example 1; 6-well plates for were used and biological duplicates were performed for all samples. Secreted alkaline phosphatase (SEAP) activity (expressed as U/ml of media) was assayed 9 hours after transfection using AbCam commercial kit.

To ensure equal transfection efficiency across the experiment, a co-transfection with CMV-eGFP vector was performed (10% of the DNA mass used for transfection was linear DNA encoding eGFP). Median fluorescence was measured using flow cytometry and confirmed to be invariant across the experiment.

Reduced amounts of the vector with the SEAP reporter gene were used while the competitor vector (no reporter gene) maintains constant mass of DNA transfected.

TABLE 7
Transfection protocol at reduced amount of vector (0.4 μg/well)
DNA dilution in media (Mix A)

| Conc. | | Volume | Total |
|---|---|---|---|
| (μg/mL) | DNA construct | (μl) | DNA (μg) |
| 125 | eGFP standard | 2 | 0.25 |
| 125 | competitor vector | 10 | 1.25 |
| 125 | Vectors with binding | 8 | 1.00 |
| | motifs and SEAP | | |
| | DMEM media | 230 | |
| | total | 250 | 2.50 |

PEIpro dilution in media (Mix B)

| Solution | Volume (μl) |
|---|---|
| PEIpro | 10 |
| DMEM media | 240 |
| total | 250 |

Transfection mix (for 2.5 wells)

| Solution | Volume (μl) |
|---|---|
| Mix A (DNA) | 250 |
| Mix B (PEIpro) | 250 |
| Total | 500 |

Add to each well

| Volume (μL) | Total DNA (μg) |
|---|---|
| 200 | 1.00 |

TABLE 8
Transfection protocol at reduced amount of vector (0.2 μg/well)
DNA dilution in media (Mix A)

| | | Volume | Total |
|---|---|---|---|
| Conc. (μg/mL) | DNA construct | (μl) | DNA (μg) |
| 125 | eGFP standard | 2 | 0.25 |
| 125 | competitor vector | 14 | 1.75 |
| 125 | Vectors with | 4 | 0.50 |
| | binding motifs and | | |
| | SEAP | | |
| | DMEM media | 230 | |
| | total | 250 | 2.50 |

PEIpro dilution in media (Mix B)

| Solution | Volume (μl) |
|---|---|
| PEIpro | 10 |
| DMEM media | 240 |
| total | 250 |

Transfection mix (for 2.5 wells)

| Solution | Volume (μl) |
|---|---|
| Mix A (DNA) | 250 |
| Mix B (PEIpro) | 250 |
| Total | 500 |

Add to each well

| Volume (μl) | Total DNA (μg) |
|---|---|
| 200 | 1.00 |

>hTEL:
ctgcgcgctcgctcgctcactgaggcctttagggttagggttagggttagg
gttggcctcagtgagcgagcgagcgcgcag

Results

The expression levels of vectors that include the structural motif hTel are significantly greater than of the reference vector (no apt) when lower amounts of the vectors are used in the transfection—0.4 μg/well (FIG. 2) and 0.2 μg/well (FIG. 3). This demonstrates that the vector may be used in lower amounts than unmodified vectors.

### Example 3

Nuclear Import Enhancement Assessed in HepG2 Cell Line

The vectors as described above were transfected to HepG2 using PElpro transfection reagent following the manufacturer recommendations, as outlined in Example 1. 6-well plates for were used and biological duplicates were performed for all samples. Secreted alkaline phosphatase (SEAP) activity (expressed as U/ml of media) was assayed 9 hours after transfection using AbCam commercial kit. To ensure equal transfection efficiency across the experiment, a co-transfection with CMV-eGFP vector was performed (10% of the DNA mass used for transfection was linear DNA encoding eGFP). Median fluorescence was measured using flow cytometry and confirmed to be invariant across the experiment.

TABLE 9
Transfection protocol at reduced amount of vector
DNA dilution in media (Mix A)

| Conc. | | Volume | Total |
|---|---|---|---|
| (ug/ml) | DNA construct | (μl) | DNA (μg) |
| 125 | eGFP standard | 2 | 0.25 |
| 125 | competitor vector | 10 | 1.25 |
| 125 | Vectors with binding | 8 | 1.00 |
| | motifs and SEAP | | |
| | DMEM media | 230 | |
| | total | 250 | 2.50 |

PEIpro dilution in media (Mix B)

| Solution | Volume (μl) |
|---|---|
| PEIpro | 10 |
| DMEM media | 240 |
| Total | 250 |

Transfection mix (for 2.5 wells)

| Solution | volume (μl) |
|---|---|
| Mix A (DNA) | 250 |
| Mix B (PEIpro) | 250 |
| Total | 500 |

Add to each well

| Volume (μl) | Total DNA (μg) |
|---|---|
| 200 | 1.00 |

>hTEL
ctgcgcgctcgctcgctcactgaggcctttagggttagggttagggttagg
gttggcctcagtgagcgagcgagcgcgcag

Results SEAP expression from vectors including the structural motif hTel in HepG2 cell culture is significantly greater than the equivalent vector lacking the structural motif (no apt), FIG. 4.

### Example 4

Demonstration that the Binding Motif is Functional in the Vector—Streptavidin Aptamers

To ensure that the end conversion method for synthesizing the vectors is compatible with independent folding of the structure to form the capped ends, binding of streptavidin aptamer to streptavidin-coated plate was chosen. Streptavidin aptamer sequence has been used downstream to the expression cassette with a mammalian reporter cassette. Two different configurations of the structural motif were synthesized including for processing on a single end of the vector: i) a single streptavidin aptamer, and ii) array of four branched streptavidin aptamers.

Sequences of processing conformational motifs with the streptavidin aptamer

>strSQ (4 aptamers)
ctgctcacctgccagctacggacgcggccacgaacgcaccgatcgcaggtttcgtggcgcgcgtaacgcaccgatcgcaggtttacgcgcagcg
agcaacgcaccgatcgcaggtttgctcgccgcccaaacgcaccgatcgcaggttttgggcgcgcgtccgtagctggcaggtgagcag
>strApt (single aptamer)
ctgctcacctgccagctacggacgcggggaacgcaccgatcgcaggtttccccgcgtccgtagctggcaggtgagcag
>noApt (no aptamer control)
ctgctcacctgccagctacggacgcggaacgcgtccgtagctggcaggtgagcag
Duplex sequence
>SEAP-2A-eGFP
cgttacataacttacggtaaatggcccgcctggctgaccgcccaacgacccccgcccattgacgtcaataatgacgtatgttcccatagtaacgc
caatagggactttccattgacgtcaatgggtggagtatttacggtaaactgcccacttggcagtacatcaagtgtatcatatgccaagtacgcccc
ctattgacgtcaatgacggtaaatggcccgcctggcattatgcccagtacatgaccttatgggactttcctacttggcagtacatctacgtattag
tcatcgctattaccatggtgatgcggttttggcagtacatcaatgggcgtggatagcggtttgactcacggggatttccaagtctccaccccattg
acgtcaatgggagtttgttttggcaccaaaatcaacgggactttccaaaatgtcgtaacaactccgccccattgacgcaaatgggcggtaggcgtg
tacggtgggaggtctatataagcagagctcctaggcgtttagtgaaccgtcagaatcgatcgaatcccggccgggaacggtgcattggaacgcgg
attccccgtgccaagagtgacgtaagtaccgcctatagagtctataggcccacaaaaaatgctttcttcttttaatatacttttttgtttatctta
tttctaatactttccctaatctctttctttcagggcaataatgatacaatgtatcatgcctctttgcaccattctaaagaataacagtgataattt
ctgggttaaggcaatagcaatatttctgcatataaatatttctgcatataaattgtaactgatgtaagaggtttcatattgctaatagcagctaca
atccagctaccattctgcttttattttatggttgggataaggctggattattctgagtccaagctaggcccttttgctaatcatgttcatacctct
tatcttcctcccacagctcctgggcaacgtgctggtctgtgtgctggcccatcactttggcaaagaattgggatatcgattgatggctgtaagctt
ggaccgccaccatggtgagcaagggcgaggagctgttcaccggggtggtgcccatcctggtcgagctggacggcgacgtaaacggccacaagttca
gcgtgtccggcgagggcgagggcgatgccacctacggcaagctgaccctgaagttcatctgcaccaccggcaagctgcccgtgccctggcccaccc
tcgtgaccaccctgacctacggcgtgcagtgcttcagccgctaccccgaccacatgaagcagcacgacttcttcaagtccgccatgcccgaaggct
acgtccaggagcgcaccatcttcttcaaggacgacggcaactacaagacccgcgccgaggtgaagttcgagggcgacaccctggtgaaccgcatcg
agctgaagggcatcgacttcaaggaggacggcaacatcctggggcacaagctggagtacaactacaacagccacaacgtctatatcatggccgaca
agcagaagaacggcatcaaggtgaacttcaagatccgccacaacatcgaggacggcagcgtgcagctcgccgaccactaccagcagaacacc
cccatcggcgacggccccgtgctgctgcccgacaaccactacctgagcacccagtccgccctgagcaaagaccccaacgagaagcgcgatcac
atggtcctgctggagttcgtgaccgccgccgggatcactctcggcatggacgagctgtataagggaagcggagctactaacttcagcctgctgaa
gcaggctggagacgtggaggagaaccctggacctatggttctggggccctgcatgctgctgctgctgctgctgctgggcctgaggctacagctct
ccctgggcatcatcccagttgaggaggagaacccggacttctggaaccgcgaggcagccgaggccctgggtgccgccaagaagctgcagcctg
cacagacagccgccaagaacctcatcatcttcctgggcgatgggatgggggtgtctacggtgacagcagccaggatcctaaaagggcagaaga
aggacaaactggggcctgagatacccctggctatggaccgcttcccatatgtggctctgtccaagacatacaatgtagacaaacatgtgccaga
cagtggagccacagccacggcctacctgtgcggggtcaagggcaacttccagaccattggcttgagtgcagccgcccgctttaaccagtgcaac
acgacacgcggcaacgaggtcatctccgtgatgaatcgggccaagaaagcagggaagtcagtgggagtggtaaccaccacacgagtgcagca
cgcctcgccagccggcacctacgcccacacggtgaaccgcaactggtactcggacgccgacgtgcctgcctcggcccgccaggaggggtgcca
ggacatcgctacgcagctcatctccaacatggacattgatgtgatcctgggtggaggccgaaagtacatgtttcgcatgggaaccccagaccctg
agtacccagatgactacagccaaggtgggaccaggctggacgggaagaatctggtgcaggaatggctggcgaagcgccagggtgcccggtat
gtgtggaaccgcactgagctcatgcaggcttccctggacccgtctgtgacccatctcatgggcctctttgagcctggagacatgaaatacgagat
ccaccgagactccacactggacccctccctgatggagatgacagaggctgccctgcgcctgctgagcaggaacccccgcggcttcttcctcttcg
tggagggtggtcgcatcgaccacggtcatcacgaaagcagggcttaccgggcactgactgagacgatcatgttcgacgacgccattgagaggg
cgggccagctcaccagcgaggaggacacgctgagcctcgtcactgccgaccactcccacgttttctccttcggaggctaccccctgcgagggag
ctccatcttcgggctggcccctggcaaggcacgggacaggaaggcctacacggtcctcctatacggaaacggtccaggctatgtgctcaaggac
ggcgcccggccggatgttaccgagagcgagagcgggagccccgagtatcggcagcagtcagcagtgcccctggacgaagagacgcacgcag
gcgaggacgtggcggtgttcgcgcgcggcccgcaggcgcacctggttcacggcgtgcaggagcagaccttcatagcgcacgtcatggccttcgc
cgcctgcctggagccctacaccgcctgcgacctggcgccccccgccggcaccaccgacgccgcgcacccagggcggtcccggtccaagcgtctg
gattgagaattccctttcggggcagacatgataagatacattgatgagtttggacaaaccacaactagaatgcagtgaaaaaaatgctttatttg
tgaaatttgtgatgctattgctttatttgtaaccattataagctgcaataaacaagtt

Obtained vectors were tested for binding a streptavidin-coated plate. The amount of DNA retained on the plate was detected by pico-green incorporation assay.

Binding Protocol

DNA was mixed with streptavidin-coated plate in binding buffer for 2 h, at room temp. Plate was washed 3× with same binding buffer, and the DNA that was bound to the plate was detected with PicoGreen assay in plate, without detaching DNA.
Binding buffer (10×)
1 M NaCl
20 mM MgCl2
50 mM KCl
10 mM CaCl2)
200 mM Tris-HCl, pH 7.6

Result

Vectors according to the present invention which comprise of four streptavidin aptamers (circle) binds significantly stronger (dissociation constant, KD=5.6 nM) than a vector containing a single aptamer (KD=17.4 nM, square). Control DNA with no aptamer on at either end showed no specific binding to the plate. The result constitutes the evidence that functional aptamers can be located at the end of the vector as a single motif or array of motifs. Fitting curve is shown in FIG. 8.

### Example 5

Production of Vector with Custom Capped Ends, Covalently Closed.

Template: Template A (FIG. 11).

The template includes a nicking site, a processing motif adjacent to a conformational motif, a sequence of interest, a second conformational motif adjacent to a second processing motif, and a backbone of similar size to the sequence of interest. There is an additional endonuclease target site in the backbone, which will only cut in dsDNA.

Nicking Reaction in 20 μl
4 μl template (1 μg/μl)
13 μl H₂O
2 μl CutSmart buffer (NEB)
1 μl nickase (Nb.BsrDl, NEB)
Incubated for 180 minutes at 37° C., then 20 minutes at 80° C.

Amplification Reaction in 1000 μl
4 μl template (0.2 μg/μl)
100 μl buffer—10×
   300 mM Tris pH 7.9
   300 mM KCl
   50 mM (NH₄)₂SO₄
   100 mM MgCl₂
837 μl ddH₂O
20 μl dNTPs (100 mM) (Bioline)
35 μl SSB (5 μg/μl) (E. coli SSB, in-house preparation)
2 μl inorganic pyrophosphatase (2 U/μl) (Enzymatics)
2 μl phi29 DNA polymerase (100 U/μl) (Enzymatics)
Incubated for 16 hours at 30° C.

Processing Reaction
1000 μl amplification reaction
20 μl Mlyl (10 U/μl)
Incubated for 180 minutes at 37° C.

Purification Reaction

200 μl of the processed reaction was run through a PCR clean-up column (Macherey-Nagel) and eluted in 20 μl

Second-Strand Synthesis Reaction in 50 μl
10 μl template
1 μl T4 DNA polymerase (exo⁻) (3 U/μl)
1 μl T4 DNA ligase (400,000 U/μl)
5 μl T4 DNA ligase buffer @ 10×
   50 mM Tris-HCl
   −10 mM MgCl₂
   1 mM ATP
   −10 mM DTT
0.5 μl dNTPs (40 mM)
32.5 μl ddH₂O
Incubated for 180 minutes at 37° C.

Exonuclease Clean-Up
25 μl second-strand synthesis reaction
0.2 μl T5 exonuclease (10 U/μl)
Incubated for 16 hours at 37° C.

Result: Gel shown in FIG. 12. FIG. 12 shows a 0.8% agarose gel stained with SafeView demonstrating production of closed linear DNA vectors by second-strand synthesis and ligation.

Lanes 1 & 9 are Thermo Scientific Gene Ruler 1 kb Plus DNA ladder. Lane 2 lacks all enzymes; lane 3 includes T4 DNA ligase; lane 4 includes T4 DNA ligase and the T5 exonuclease clean-up step; lane 5 includes T4 DNA polymerase; lane 6 includes T4 DNA polymerase and the T5 exonuclease clean-up step; lane 7 includes both T4 polymerase and T4 ligase; lane 8 includes both T4 polymerase and T4 ligase and the T5 exonuclease step.

It can be seen that in the presence of only one of the two enzymes, no exonuclease-resistant (i.e. closed DNA) products are present, but including both polymerase and ligase results in the formation of a closed molecule which resists exonuclease degradation.

### Example 6

Nuclear Import May be Enhanced by the Presence of a Multiple Binding Motifs at the 3′-End (Cap) of the Vector.

In order to examine the effect of including an array of binding motifs in one structural motif, several nuclear import experiments were conducted, using binding motifs that targeted various nuclear elements such as histone and nucleolin.

Linear double stranded DNA with a mammalian expression cassette [Ef1a-SEAP-SV40poly(A)] has been generated to contain different versions multiple aptamers on its 3′-end to target:
i) human Telomere G-quadruplex—histone H4 (hTel—H4_Gq)
ii) 3×nucleolin (3×nucl)
iii) histone H4—nucleolin (H4_Gq—nucl)
iv) 4× EpCAM stem loop (4× EpCAM)

DNA was transfected into HEK293T using PEI Pro transfection reagent. Secreted alkaline phosphatase (SEAP) activity (expressed as U/mL of media) was assayed 9 hours after transfection using AbCam commercial kit (AbCam, Cambridge, UK). To ensure equal transfection efficiency across the Example, a co-transfection with CMV-eGFP vector was performed (10% of the DNA mass used for transfection was linear DNA encoding eGFP). Median fluorescence was measured using flow cytometry and confirmed to be invariant across the experiment.

Sequences used:

>hTel-H4_Gq
ctgctcacctgccagctacggacgcggccacgtttagggttagggttagggttagggttcgtggcagcgcgttggtggggttcccgggagg
gcggctacgggttccgtaatcagatttgtgtacgcgccgcgtccgtagctggcaggtgagcag
>3x nucl
ctgctcacctgccagctacggacgcggccacgtggtggtggtggttgtggtggtggtgggcgtggcagcgcgttggtggtggtggttgtgg
tggtggtgggacgcgcagcgagctggtggtggtggttgtggtggtggtggggctcgccgcgtccgtagctggcaggtgagcag
>H4_Gq-nucl
ctgctcacctgccagctacggacgcggccacgtggtggggttcccgggagggcggctacgggttccgtaatcagatttgtgtcgtggcagc
gcgttggtggtggtggttgtggtggtggtgggacgcgccgcgtccgtagctggcaggtgagcag
>4xEpCAM
ctgctcacctgccagctacggacgcggccacgACAGAGGTTGCGTCTGTcgtggcgcgcgtACAGAGGTTGCGTCTGTacgcgc
agcgagcACAGAGGTTGCGTCTGTgctcgccgcccaACAGAGGTTGCGTCTGTtgggcgcgcgtccgtagctggcaggtgagc
ag

The results for this Example are depicted in FIG. 13, which clearly shows that including an array of binding motifs is advantageous during nuclear targeting. The control DNA (“No Apt”) clearly produced less SEAP than all of the experimental versions including an array of nuclear targeting binding motifs. Multiple aptamers clustered within a single structural motif can be seen to have up to a fivefold increase in reporter gene expression. In order to ensure proper folding of the array, branching stems of unique sequences have been designed to enforce the independent folding of each of motif and to limit the fold of the array to a single possible conformation.

1. A targeting DNA expression vector including a duplexed section of DNA, characterised in that the duplexed section is capped at both ends, wherein at least one end of the duplex is capped with a structural motif and said structural motif includes at least one binding motif which forms a conformation capable of binding to a cellular target.

2. The targeting expression vector of claim 1 wherein the duplex DNA is capped at both ends with a structural motif, which can be the same or different.

3. The targeting expression vector of claim 1 wherein the duplex DNA is capped at one end with a structural motif and at the second end with a hairpin, a T shaped hairpin, a cross-arm, a stem loop, a loop, a bulge or a cruciform.

4. The targeting expression vector of any previous claim wherein said structural motif includes an array of binding motifs, which can be the same or different.

5. The targeting expression vector of any previous claim wherein the binding motif is capable of binding to a cellular target on any one or more of:
(i) a cell surface;
(ii) the nuclear envelope;
(iii) the nuclear transport system;
(iv) a cellular compartment
(v) a nuclear component;
(vi) a cytoplasmic inclusion; and/or
(vii) a cytoplasmic protein or peptide

6. The targeting expression vector of any one of claims 1 to 5 wherein said vector also includes a binding motif capable of binding to any one or more of:
(i) a peptide or protein;
(ii) a small molecule;
(iii) an antibody or derivative thereof;
(iv) an enzyme;
(v) an immunostimulant
(vi) an agonist or antagonist;
(vii) an adjuvant and/or
(viii) nucleic acid.

7. The targeting expression vector of claim 5 wherein said target is present in or on a eukaryotic cell, optionally a plant cell, protist cell, fungal cell, human cell or a non-human animal cell.

8. The targeting expression vector of claim 5 wherein said target is present on or in a prokaryotic cell, optionally wherein said cell is a bacterial cell.

9. The targeting expression vector of any preceding claim wherein said linear duplex DNA includes a gene sequence or a fragment thereof and optionally a promoter.

10. The targeting expression vector of claim 9 wherein said gene or fragment thereof encodes a functional RNA molecule.

11. The targeting expression vector of any preceding claim wherein said vector includes modified nucleotides, optionally modified nucleotides in the capped ends.

12. The targeting expression vector of any preceding claim wherein the expression vector is substantially pure DNA, optionally 95% DNA.

13. The targeting expression vector of any preceding claim wherein the structural motif permits the formation of hydrogen bonds between the nucleotide bases in the sequence of the structural motif, optionally wherein said hydrogen bonds between the nucleotide bases involve Watson-Crick base pairs, Hoogsteen base-pairs or non-canonical base-pairing

14. The targeting expression vector of any preceding claim wherein one or both of the capped ends are covalently closed.

15. The targeting expression vector of any preceding claim wherein said structural motif forms a non-canonical DNA structure, and may include any one or more of:
a) a hairpin;
b) a cross-arm;
c) a triplex;
d) a G-triplex;
e) a G quadruplex;
f) an i-motif;
g) a pseudoknot;
h) a stem loop; and/or
i) a bulge or loop.

16. The targeting expression vector of any preceding claim wherein the structure the binding motif assumes permits association with the target in a structure and/or sequence-dependent manner.

17. The targeting expression vector of any preceding claim wherein the binding motif is any one or more of:
a) an aptamer;
b) a quadruplex;
c) a catalyst;
d) an i-motif; and/or
e) triple stranded DNA.

18. The targeting expression vector of any preceding claim wherein the binding motif is specific.

19. A method of manufacturing a vector which includes a duplexed section capped at both ends by a structural motif, comprising:
(a) provision of a nucleic acid template comprising a sequence encoding:
(i) a first processing motif, adjacent to
(ii) a first structural motif,
(iii) a single strand of said duplex DNA,
(iv) a second structural motif, adjacent to
(v) a second processing motif
said processing motif includes a sequence capable of forming a base-paired section including a recognition site for an endonuclease containing a cleavage site,
said structural motif includes at least one sequence capable of forming intramolecular hydrogen bonds and forming a capped end,
and optionally either of said first or second capped ends includes a binding motif;
(b) amplifying said template using a polymerase capable of rolling circle amplification such that a single stranded concatemer is produced;
(c) contacting the concatemer with an endonuclease to release single stranded DNA intermediates wherein the 3′ terminal nucleotide is base paired adjacent to a single stranded portion of the construct; and
(d) contacting the single stranded DNA constructs with a polymerase enzyme to extend the 3′ terminal nucleotide using the single stranded DNA intermediate as a template to form the duplex section.

20. The method of claim 19 wherein said 5′ terminal nucleotide is base paired adjacent to a single stranded portion of the vector and said method further comprises the use of a ligase enzyme to covalently close the vector.

21. The nucleic acid of claim 1 wherein the binding motif includes the presence of specific nucleotide residues within the conformation to permit binding to a cellular target.

22. The nucleic acid of claim 1 wherein the binding motif binds to a cellular target that is selected from a protein; a modified protein including a glycoprotein, a lipoprotein; a peptide; a carbohydrate; a lipid or a modified lipid including a glycolipid or a phospholipid.
